(19) United States
(12) Patent Application Publication
(10) Pub. No.: US 2004/0197316 A1
(43) Pub. Date: Oct. 7, 2004

(54) Novel method for the treatment of systemic lupus erythematosus

(76) Inventors: George C. Tsokos, Silver Spring, MD (US); Yuang-Taung Juang, Potomac, MD (US)

Correspondence Address: NASH & TITUS, LLC, 21402 UNISON RD, MIDDLEBURG, VA, 20117, US

(21) Appl. No.: 10/772,704

(22) Filed: Feb. 5, 2004

Related U.S. Application Data
(60) Provisional application No. 60/445,397, filed on Feb. 6, 2003.

(51) Int. Cl. A61K 48/00

(52) U.S. Cl. 424/93.21

(57) ABSTRACT

A method for the treatment of systemic lupus erythematosus (SLE) by administering antisense cAMP response element modulator (antisense CREM) to patients with systemic lupus erythematosus. The antisense CREM increases the production Interleukin-2 (IL-2) which is decreased in SLE patients. Additionally, this invention relates to taking freshly isolated SLE T cells and transfecting them with TCR ζ chain construct in a eukaryotic expression vector, at high efficiency by a recently developed nucleoporation technique for the restoration of TCR/CD3-mediated signaling in the ζ chain transfected cells. Reconstitution of deficient TCR ζ chain can reverse the TCR/CD3-mediated signaling abnormalities as well as the defective IL-2 production in T cells of SLE patients.

This invention claims priority under 35 USC §119 of provisional application serial No. 60/445,397 filed Feb. 6, 2003.

### GOVERNMENT INTEREST

The invention described herein may be manufactured, used and licensed by or for the U.S. Government.

### BACKGROUND OF THE INVENTION

1. Field of the Invention

This invention relates to identifying missing and/or over-expressed gene products in patients with systemic lupus erythematosus (SLE) and re-introducing genes that code for these proteins or genes that would suppress the expression of inappropriately over-expressed genes into T cells from SLE patients to restore normal immune cell function. This is accomplished by leukopheresis of lymphocytes from patients with SLE, transfecting them with appropriate vector(s) and re-infusing them into the same patients to treat the condition.

More specifically, this invention relates to a method for the treatment of systemic lupus erythematosus (SLE) by administering antisense cAMP response element modulator (antisense CREM) to patients with systemic lupus erythematosus. The antisense CREM increases the production Interleukin-2 (IL-2) which is decreased in SLE patients.

Additionally, this invention relates to taking freshly isolated SLE T cells and transfecting them with TCR ζ chain construct in a eukaryotic expression vector, at high efficiency by a recently developed nucleoporation technique for the restoration of TCR/CD3-mediated signalling in the ζ chain transfected cells. Reconstitution of deficient TCR ζ chain can reverse the TCR/CD3-mediated signalling abnormalities as well as the defective IL-2 production in T cells of SLE patients.

In addition, in the context of this invention, we will introduce small interference RNA (siNA) to suppress the expression of CREM in SLE T cells.

2. Brief Description of Related Art

Systemic lupus erythematosus (also called SLE or lupus) causes a variety of problems. It may cause skin rashes, arthritis, anemia, seizures or psychiatric illness, and often affects internal organs including the kidneys, lungs and heart. Once a disease with high mortality, SLE is now considered a chronic disease.

SLE is a chronic inflammatory disorder resulting from an abnormality of the immune system, which normally functions to protect the body against cancers and invading infections. In SLE, the immune system is over-active and produces too many abnormal antibodies that react with the patient's own tissues. The exact cause of lupus is not known, but heredity, environment and hormonal changes may be involved.

Prevalence of SLE is 40 to 50 per 100,000. It is more common in certain ethnic groups, particularly among blacks. More than 85 percent of lupus patients are women. Because of its wide variety of symptoms, diagnosis is often difficult and requires a high degree of awareness among physicians. Typical features of SLE include: 1. A butterfly-shaped rash over the cheeks. 2. A skin rash appearing in areas exposed to the sun. 3. Sores in the mouth and nose. 4. Arthritis involving one or more joints. 5. Kidney inflammation. 6. Nervous system disorders including seizures, mental disorders, and strokes. 7. Fever, weight loss, hair loss, poor circulation in the fingers and toes, chest pain when taking deep breaths (pleurisy) and abdominal pain are often seen. 8. People with lupus are more likely to have clogged arteries that can lead to heart attack and stroke at a younger age, likely due to the inflammation from lupus.

Medications cannot cure systemic lupus erythematosus (SLE), but only control symptoms in an attempt to prevent or slow organ damage. Because most SLE symptoms are caused by inflammation, first-line medications are anti-inflammatory, such as nonsteroidal anti-inflammatory drugs (NSAIDs) and antimalarials.

A person having mild disease or symptoms that affect quality of life but does not have life-threatening organ problems, may be treated with nonsteroidal anti-inflammatory drugs (NSAIDs) or antimalarial drugs (such as hydroxychloroquine Plaquenil).

Antimalarial medications for systemic lupus erythematosus include hydroxychloroquine sulfate (Plaquenil) and chloroquine hydrochloride (Aralen). These antimalarial medications are not labeled by the U.S. Food and Drug Administration (FDA) for the treatment of lupus but are often prescribed for people with lupus to reduce inflammation.

The most significant side effect of antimalarials is damage to the tissue that lines the eye (retina); this is rare when appropriate doses are used. Regular eye examinations with your doctor and possibly at home are critical to preventing eye damage. An initial eye examination will be done if you are taking hydroxychloroquine (Plaquenil) or chloroquine (Aralen) for more than 3 months. Other side effects include occasional rash, nausea, or diarrhea.

Hormonal therapy is another form of treatment for symptoms of systemic lupus erythematosus. Hormone therapies for SLE include DHEA (dehydroxyepiandrosterone). DHEA is an androgenic dietary supplement that is derived from the wild yam; only pharmaceutical-grade (versus “natural”) DHEA is considered effective. Research suggests that it can improve stamina and sense of well-being when used to treat people with autoimmune diseases. People with SLE have reportedly had a decrease in symptoms when using DHEA. The most common side effects are acne and facial hair growth in women and hair loss in men.

Corticosteroids, the single most prescribed drugs to treat SLE, must be used judiciously. Bone protection is important when steroids are used. Common prescription corticosteroids include prednisone, dexamethasone, and hydrocortisone. Corticosteroids have many side effects, including weight gain, stomach ulcers, sleeping difficulties, increased blood pressure, increased blood sugar (glucose), delayed wound healing, and a reduced ability to fight infection. Other problems associated with corticosteroid use include cataract formation, decreased blood flow to the hip joint causing deterioration of the joint (aseptic necrosis), and osteoporosis.

Medication treatment for SLE often involves reaching a balance between preventing severe, possibly life-threatening organ damage, maintaining an acceptable quality of life, and minimizing side effects.

The medications of the past that have been used to treat SLE treat the symptoms only. None of the medications discussed above treat a cause of SLE.

The present inventors have sought to treat the cause of SLE. One of the causes of SLE has been found by the inventors to be present at the molecular level. The inventors have found that SLE patients have decreased levels of the beneficial cytokine, Interleukin-2 (IL-2) when compared to normal. The inventors have also found that IL-2 production is improved by the reconstitution of TCR ζ chain which restores T cell signalling in SLE. The reconstitution of deficient TCR ζ chain can reverse the TCR/CD3-mediated signalling abnormalities and improve the defective IL-2 production in T cells of SLE patients.

Therefore, an object of the present invention is to treat SLE at the molecular level to prevent or diminish SLE in patients.

Another object of the present invention is to increase the production of IL-2 by eliminating the presence of CREM which is overexpressed in SLE T cells and represses the transcription of the IL-2 gene and subsequently the production of IL-2 protein.

### SUMMARY OF THE INVENTION

The present invention solves the problems of the past by treating the cause of SLE at the molecular level. This is accomplished by the application of antisense CREM to SLE T cells to increase IL-2 production. IL-2 production is also improved by the reconstitution of TCR ζ chain which restores T cell signalling in SLE T cells and patients.

### BRIEF DESCRIPTION OF THE FIGURES

FIG. 1A is a digital image of an agarose gel showing the RT-PCR products of b-actin (control) and CREM from normal (NL) and SLE patients. The last two lanes show that SLE T cells have more CREM mRNA than normal T cells. The levels of the control actin mRNA are comparable in SLE and normal T cells (first 3 lanes);

FIG. 1B shows the densitometric readings of gels similar to those presented in FIG. 1a form 16 normal individuals and 18 SLE patients (marked in the abscissa). The mean densitometric readings for the SLE group are significantly higher than those of the normal group (means are marked with horizontal lines and the brackets indicate SEM);

FIG. 1C shows a digital image of an agarose gel similar to that shown in FIG. 1a taken from cells from SLE and normal individuals after they were treated with actinomycin D. The fact that the intensity of the bands remains the same indicates that the stability of the is CREM mRNA is comparable in SLE and normal T cells;

FIG. 1D shows correlation diagram between the levels of IL2 and CREM mRNA. The value of 0.56 indicates that them more CREM a T cell has, the less IL-2 it produces;

FIG. 2 shows a digital image of agarose gels of PCR products of precipitates from SLE and control (normal) T cells. The precipitates were generated with anti-CREM antibody and other control antibodies. The intense band in first lane marked SLE, indicates that a significant amount of CREM binds to the IL2 promoter in live SLE T cells;

FIG. 3 shows the mean ±SEM of the mean of luciferase activity in cells in which CEM, or anti-sense-CREM or empty vectors were introduced. This is the first demonstration that antisense CREM can enhance the activity of the IL2 promoter;

FIG. 4A shows a digital image of a Western blot from lysates form cells treated with either an empty vector or anti-sense CREM. The firs lane is from a blot with anti-CREM antibody and shows that anti-sense decreases the amounts of CREM protein. The second is from a blot with anti-CREB antibody and the third from a blot with anti beta-actin. That last two blots show that the effect in limited to CREM and therefore it is specific;

FIG. 4B is a digital image of an agarose gel of IL-2 PCR products from cells treated with anti-sense or empty vector. The increased intensity of the left lane indicates that anti-sense CREM can effectively increase the amounts of IL-2 mRNA;

FIG. 4C shows a recording of a RT PCR using primers to detect IL2 mRNA from cells treated with anti-sense CREM or empty vectors;

FIG. 5A shows that mean (±SEM) of IL-2 protein produced by cells treated with anti-sense CREM or empty vector;

FIG. 5B shows that mean (±SEM) of IL-2 protein produced by cells treated with anti-sense CREM or empty vector while they were stimulated with anti-CD3 and anti-CD28 antibodies to promote production of IL-2;

FIG. 6A (upper part) shows digital images of gels of PCR products following precipitation of parts of the IL2 promoter with anti-CREM antibody. Cells treated with ASV (anti-sense vector) displayed the lowest intensity band. The lower part shows a photo of a gel of PCR products of IL2 mRNA;

FIG. 6B shows a digital image of a gel of a shift assay showing the intensity of the protein binding to the −180 site of the IL2 promoter. The strong intensity of the right lane indicates that CREM binds intensely when cells are transfected with CREM vector. The low intensity of the middle band indicates that CREM binding is eliminated when the cells are transfected with anti-sense CREM vector;

FIG. 6C shows a digital image of a super shift gel showing that the binding that was detected in FIG. 6B is specific that is, it is eliminated in the presence of anti-CREM antibody;

FIG. 7A1-7A2 show a digital image of a recording from a flow cytometry recording showing cells transfected with the vector expressing GFP (a green color protein);

FIG. 7B shows a digital image of blots of detergent soluble lysates of cells transfected with an empty vector or a vector expressing TCR zeta chain. The antibodies used to perform the blots are shown to the right;

FIG. 7C shows a digital image of blots of detergent in-soluble lysates of cells transfected with an empty vector or a vector expressing TCR zeta chain. The antibodies used to perform the blots are shown to the right;

FIG. 8A1-8A4 show a flow cytometric recording of non-permeabilized cells stained with an anti-CD3 epsilon antibody or an isotype control after being transfected with an empty vector or a vector expressing TCR zeta chain;

FIG. 8B1-8B4 shows a flow cytometric recording of permeabilized cells stained with an anti-CD3 epsilon antibody or an anti-TCR zeta chain antibody after being transfected with an empty vector or a vector expressing TCR zeta chain;

FIG. 9A shows a digital image of a blot of lysates of cells transfected with empty vector or a vector expressing tcr zeta chain. The antibody used in the blots is an anti-phosphotyrosine one;

FIG. 9B shows a tracing of free calcium concentration of normal T cells and cells transfected either with an empty vector or with a tcr zeta chain expression vector;

FIG. 10A shows digital images of bolts of lysates of cells transfected either with an empty vector (right) or a vector expressing TCR zeta chain (left). An anti-FcR gamma antibody was used to prepare the blots;

FIG. 10B shows the mean ±SEM of densitomteric readings of bands detected in agarose gels performed as in 10a;

FIG. 10C shows digital images of agarose gels of pCR products of FcR gamma mRNA and beta actin in cells transfected either with an empty vector (right) or a vector expressing TCR zeta chain (left);

FIG. 10D shows the mean densitometric readings (±SEM) of gels run as in 10c;

FIG. 11 is a composite of digital images of cells stained with for LAT or zeta chain (first and second row). The third row represents the merging of the figures presented in the first two rows;

FIG. 12 shows the mean levels of IL2 protein (±SEM) produced by cells transfected either with an empty vector (left) or a vector expressing TCR zeta chain (right);

FIG. 13A is a digital image of bolts of cell lysates from normal (N) or SLE cells treated either with an empty vector or a vector expressing TCR zeta chain as shown. The antibodies used to prepare the blots are indicated on the right side; and

FIG. 13B show the mean densitometric readings of 3 exps performed as in 13a.

### DETAILED DESCRIPTION

The cAMP response element modulator (CREM) has been shown by the inventors to bind specifically to the −180-site of the interleukin-2 promoter in vitro. CREM protein was found increased in T cells of patients with systemic lupus erythematosus (SLE), and it is considered responsible for the decreased production of IL-2. The inventors have found that transcriptional upregulation is responsible for the increased CREM protein levels and that CREM binds to the IL-2 promoter in live SLE T cells. The inventors have found that suppression of the expression of CREM mRNA and protein by an anti-sense CREM plasmid, which was forced to express in SLE T cells by electroporation, resulted in decreased CREM protein binding to the IL-2 promoter and increased expression of IL-2 mRNA. The inventors have found that anti-sense constructs can be used to effectively eliminate the expression of a transcriptional repressor. This approach can be used therapeutically in conditions where increased production of IL-2 is desired for the treatment of SLE.

IL-2 is a critical cytokine in T cell development and maturation. It is exclusively produced by T cells upon activation and is important for initiating the immune response by promoting cell cycle progression in B cells and T cells. Furthermore, IL-2 is important in terminating the response of T cells by activating the Fas pathways, which leads to apoptosis through a process known as activation induced cell death.

IL-2 transcription is regulated by several factors, which bind to the proximal promoter within 300 bp upstream of the ATG codon (₂). These transcription factors include NF-κB, NFAT, AP1, Oct and CREB and there is evidence that all binding sites on the IL-2 promoter need to be occupied to ensure maximal transcription and production of IL-2 (_{3;4}). The CREB binding site, 180 bp upstream of the start codon, is interesting for several reasons: first, mutation of this site almost completely abolishes IL-2 production (_{5;6}); second, T cells of a CREB-deficient mice show a marked decrease in the production of IL-2 (₇) and third, it serves also as the binding site of CREM, which can act as a transcriptional repressor (_{3;5}).

SLE patients often suffer and die from overwhelming infections and the decreased IL-2 production in response to antigenic stimuli represents one of the many contributing factors (_{8;9}). Inability of SLE T cells to produce IL-2 is important because on one hand it diminishes the T cell response to antigens and on the other side it fails to promote the activation induced cell death of T cells, which is necessary to terminate the immune response (₁). This can yield to a non-specific activation of T cells, which may support the production of antibodies by B cells.

The decreased production of IL-2 in response to antigenic stimulation by SLE T cells is the result of altered transcriptional regulation of the IL-2 gene in SLE T cells. Our group has shown decreased NF-κB activity due to a reduced expression of the p65 subunit in SLE T cells (₁₀). Furthermore, we have shown that T cells of SLE patients express increased amounts of CREM protein. CREM can act as a transcriptional repressor and we have proposed that this is central for decreased IL-2 production in these cells (_{3;5}).

Like CREB, ICER and ATF1, CREM belongs to the family of cAMP responsible factors, which share a high level of sequence homology. CREM is present in many tissues and has been shown to be constitutively active in spermatocytes and in the brain (₁₁₋₁₃). The expression of various isoforms of CREM is regulated by four different promoters and alternative splicing (₁₄). These isoforms can function either as transcriptional repressors or activators, depending on the presence or the absence of the transactivating domains (Tau 1 and/or 2) (_{13;15-17}). The second promoter is responsible for the regulation of an inducible CREM (ICER), which is expressed in the brain (₁₆) and has been suggested to play a role in T cells (_{18;19}), but the CREM protein that we found upregulated in SLE T cells has a much higher molecular mass than that of the ICER protein (36 kD vs. 13 kD).

We conducted experiments to determine whether the increased expression of CREM in SLE T cells is the result of increased transcriptional activity of the CREM gene and to establish that its binding to the IL-2 promoter is responsible for the decreased production of IL-2 by SLE T cells. To this end, we demonstrate that an anti-sense CREM plasmid not only decreases the expression of CREM mRNA and protein, but also upregulates the defective expression of IL-2 in SLE T cells.

Materials and Methods:

Patients and Controls: Eighteen SLE patients were studied (16 male, 2 female, mean age 43.4±4.6 years, mean SLEDAI 4.2±3.57). All met the criteria of the American College of Rheumatologists for diagnosis of Systemic Lupus Erythematosus (₂₀). As controls, 16 healthy age and gender matched volunteers were used. Written permission was obtained from each patient.

Lymphocyte isolation: Heparinized peripheral venous blood was obtained from study subjects. T-cells were separated by Rosette-separation (Stemcell, Vancouver, Canada). Briefly, non-T cells are selected by a tetrameric antibody cocktail against CD14, CD16, CD 19, CD56 and glyA and bound to erythrocytes. These complexes are separated from the T cells by a lymphoprep gradient (Nycomed Pharma, Oslo, Norway). The purified T cells were >98% positive for CD3 as tested by flow cytometry.

Antibodies: Anti-CREM, anti-CREB, anti-Jun, anti-E actin, anti-CBF1, anti-E 47, goat anti-rabbit-HRP, goat anti-mouse-HRP-conjugated mAbs were purchased from Santa Cruz Biotechnology (Santa Cruz, Calif.).

Preparation of mRNA and cDNA, PCR and Real-Time PCR: One million T cells were used for extracting RNA (RNA Easy Mini Kit, Quiagen, Germany). RNA was quantitated and 500 ng of total RNA was used for cDNA synthesis by reverse transcription (Reverse Transcription PCR Kit, Promega, Madison, Wis.). A total of 25 to 50 ng of cDNA was used for each PCR. PCR-Primers were synthesized by Sigma Genosis (The Woodlands, Tex.).

PCR-beads were used for amplification (Pharmacia Biotechnologies, Piscataway, N.J.). Real Time PCR was carried out with a Cepheid Smart Thermocycler by adding Sybr Green to the reaction mixture. Primers used for PCR were

| | |
|---|---|
| beta-actin: | |
| | 5′ CAT GGG TCA GAA GGA TTC CT 3′, |
| reverse | 5′ AGC TGG TAG CTC TTC TCC A 3′; |
| IL-2: | |
| | 5′ CAC TAC TCA CAT TAA CCT CAA CTC CTG 3′, |
| reverse | 5′ CTG GGA AGC ACT TAA TTA TCA AGT TAG TG 3′); |
| CREM: | |
| | 5′ GAA ACA GTT GAA TCC CAG CAT GAT GGA AGT 3′, |
| reverse | 5′ TGC CCC GTG CTA GTC TGA TAT ATG 3′. |

PCR products were separated on a 1.5% agarose gel and the OD was quantitated by using QuantityOne software (Bio-Rad, Hercules, Calif.) after background subtraction from each band.

For sequencing of CREM isoforms, PCR products were excised from the gel, extracted (Gel extraction Kit, Quiagen, Germany) and cloned into a TOPO cloning system (Topo TA, Invitrogen, Carlsbad, Calif.). Plasmids were isolated from recombinant clones and sequenced on an ABI Prism sequencer.

For measurement of RNA stability normal and SLE T cells were incubated for 4 h with actinomycin D (5 μg/ml) and RNA was extracted at different time points.

Preparation of nuclear extracts, Electrophoretic Mobility Shift Assay (EMSA), immunoblotting and immunoprecipitation: Five to ten million T cells were used for preparation of nuclear extracts as previously described (6). The dsDNA probe of the −180 site (−164 to 198 bp) on the IL-2 promoter was 5′-catccattcagtcagtctttgggggt-3′ in shift and super shift assays as previously described (₆). Nuclear extracts (5 μg) were separated electrophoretically on SDS Gels and used in immunoblotting studies as previously described (₆).

Chromatin Immunoprecipitation Analysis (Chip): Five million T cells were used per investigated antibody. The cells were treated with formalin (1% final concentration) for 10 minutes, washed, lysed and sonicated. The DNA-protein complexes were immunoprecipitated with a desired antibody and extracted by protein A/G sepharose beads (Santa Cruz, Calif.). After several washing steps the crosslink between DNA and protein was reversed at 65° C., followed by protein digestion with Proteinase K and the DNA was extracted (QiaAmp DNA Extraction kit, Quiagen, Germany). The DNA was amplified with primers flanking the IL-2 promoter including the −180 site (forward 5′ CTA AGT GTG GGC TAA TGT AAC 3′, reverse 5′TGT AAA ACT GTG GGG GT 3′). DNA of approximately one million cells was used per each PCR reaction. PCR products were run on a 2% agarose gel and quantified with QuantitiyOne software.

Transfection, luciferase assays and quantitative determination of IL-2: Freshly isolated normal or SLE T cells were rested for 1 h in RPMI, 10% FBS and Phytohem-agglutinin (1 μg/ml). Jurkat cells were transfected without previous stimulation with PHA. Plasmids encoding the IL-2 promoter luciferase construct (from −575 to +57 bp, a kind gift from Dr. Rao, Harvard University, Boston, Mass.), CREM α sense and anti-sense (a kind gift from Dr. Sassone-Corsi, Institut de Génétique et de Biologie Moléculaire et Cellulaire, B. P. 10142, 67404 Illkirch-Strasbourg, France) and correspponding empty vector plasmid were used for transfection. Five μg of each plasmid were used per transfection. About 5-10×10⁶ T cells were transfected by electroporation at 250 mV and 1000 μF in Opti-MEM (Gibco-BRL) and resuspended in AIMV medium (Gibco-BRL) containing 10% autologous plasma. After 20 h, T cells were harvested and the luciferase assay was carried out as described previously (⁵). IL-2 production was measured in culture supernatants by ELISA (R&D Systems, Minneapolis). For stimulation of T cells, CD 3 antibody (final concentration 10 μg/ml), CD 28 antibody (final concentration 2.5 μg/ml) and a goat-anti mouse crosslink antibody (final concentration 25 μg/ml) were used and T cells were stimulated beginning 18 hours after transfection for 6 hours.

Results:

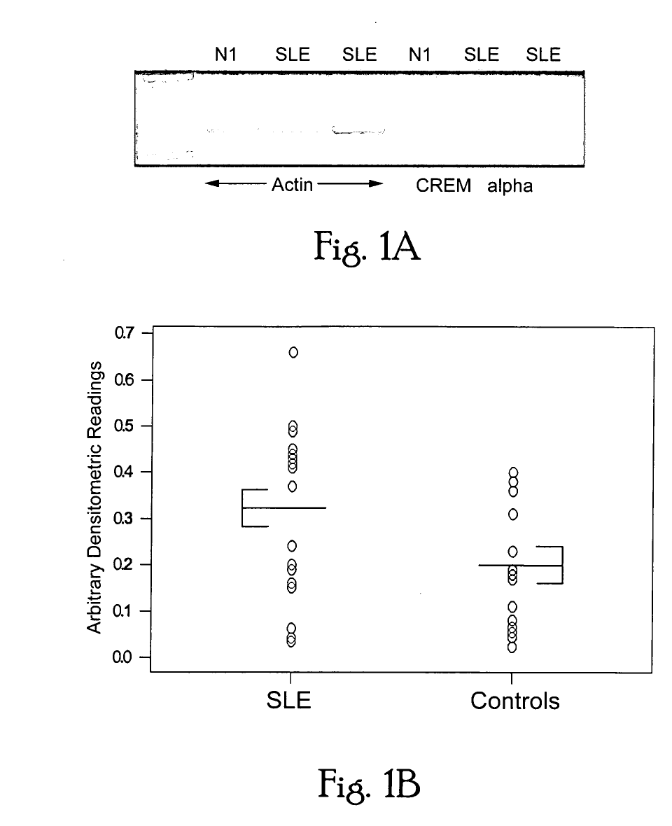

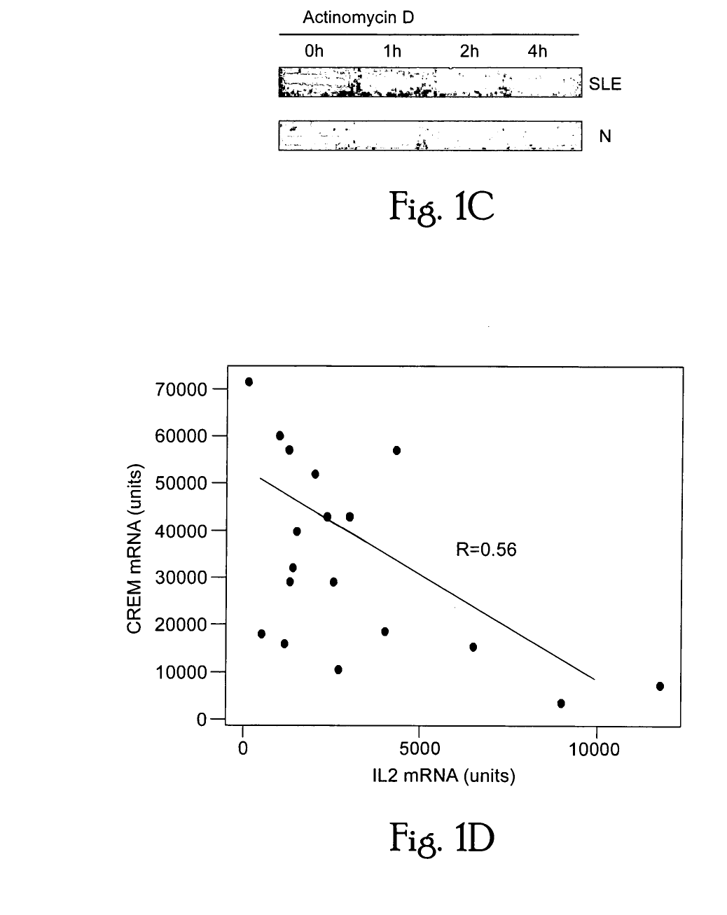

CREM is transcriptionally upregulated in SLE T cells. We have found that SLE T cells express increased amounts of the transcriptional repressor CREM protein which binds specifically at the −180 site of the IL-2 promoter, associates with the transcriptional cofactor p300 and actively suppresses the transcription of the IL-2 gene (5). To determine whether CREM was upregulated on the gene transcription level in SLE T cells, we designed appropriate oligonucleotide primers and determined the level of IL-2 and CREM mRNA by Real Time PCR. β actin primers were used as internal control. As shown in FIGS. 1A and 1B, we found a statistically significant difference of the CREM/β actin mRNA ratio between T cells from 18 SLE patients and T cells of 16 healthy controls. The RNA-stability in both SLE and normal T cells are comparable (FIG. 1C) and does not contribute to the increased amount of CREM mRNA found in SLE T cells.

We cloned and sequenced the PCR products to determine the CREM isoforms that were expressed in SLE T cells. The most dominant form of CREM mRNA in SLE T cells was the transcriptional repressor CREM α (data not shown). These data show that CREM α is transcriptionally upregulated in SLE T cells. As with a previously studied cohort of SLE patients (₅) no relationship was detected between disease activity and treatment status.

Levels of CREM mRNA correlate inversely with the IL-2 mRNA in SLE T cells. To determine whether there is a relationship between CREM α mRNA levels and IL-2 mRNA which would infer the involvement of CREM a in the regulation of the expression of IL-2, we plotted the ratio of IL-2/β actin against that of CREM α/β actin mRNA all of which had been generated by using real time PCR and visualized and quantitated on an agarose gel by methods standard in the art. As shown in FIG. 1C we noted an inverse correlation (r=0.56, p=0.014) between the levels of IL-2 and CREM α mRNA in unstimulated SLE T cells. This indicates that increased expression of CREM α mRNA is associated with decreased expression of IL-2 mRNA.

FIGS. 1a-d show increased level of CREM α mRNA in SLE T cells. Total RNA was isolated from 1×10⁶ cells, reverse transcribed and CREM, IL-2 and β actin were amplified with specific primers. The PCR products (10 μl) were electrophoresed in 1.5% agarose gels and visualized by ethidium bromide staining. A. Representative experiment of total 18 SLE patients and 16 controls: Lane 1, 100 bp DNA ladder molecular weight marker, lanes 2 and 5 control T cell samples, lanes 3, 4, 6, 7, SLE T cell samples. B. The intensity of the bands was measured by densitometry, the background subtracted and the CREM/β actin ratio calculated. Means and SEM are indicated by the horizontal and vertical lines respectively (p=0.004). C. For measurement of RNA stability normal and SLE T cells were incubated for 4 h with actinomycin D (5 μg/ml) and RNA was extracted at different time points (0, 1, 2, and 4 hours). The RNA-stability does not differ significantly between both groups. D. IL-2 mRNA/β actin ratio was plotted against the CREM α/β actin ratio mRNA (r=0.56, p=0.014).

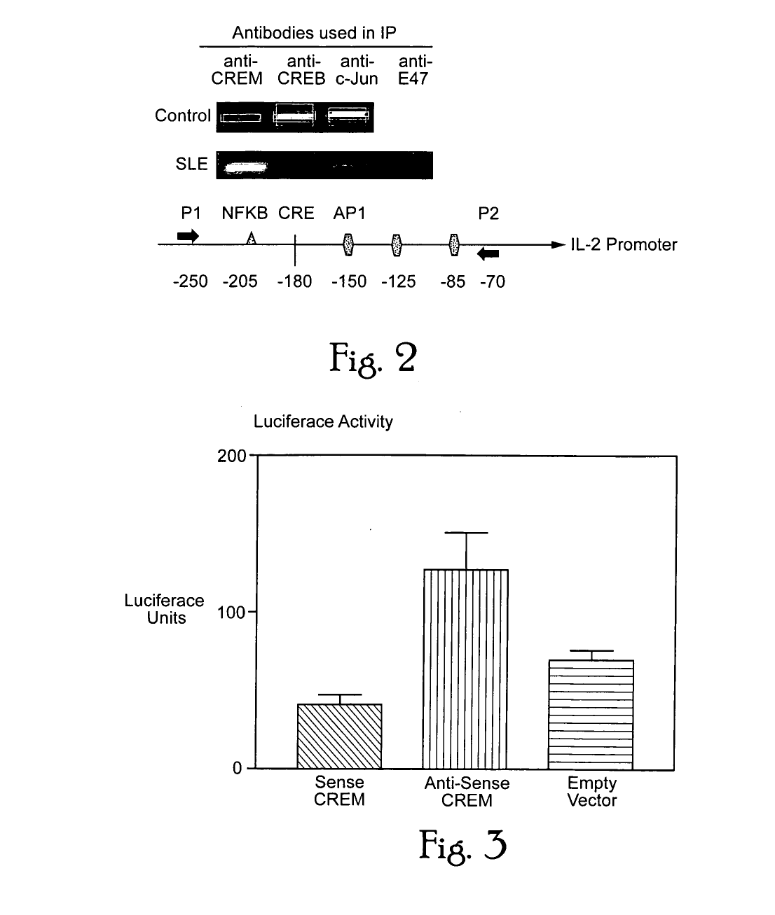

CREM binds to the IL-2 promoter in vivo. The above data, as well as those published previously (_{3;5}) have provided only indirect evidence on the significance of CREM in the repression of IL-2 expression. To determine whether CREM binds to the IL-2 promoter in live T cells we performed CHIP analysis. The cells were fixed with formalin, sonicated to break DNA to 200-300 bp fragments and incubated with appropriate antibodies to precipitate DNA-protein complexes. The immunoprecipitated DNA was extracted and detected with appropriate primers. As shown in FIG. 2 we detected increased binding of CREM to the IL-2 promoter in SLE T cells compared to normal T cells. The CREB and c-Jun binding is decreased compared to normal cells. E 47 Ab was used as control, because it does not have any known binding site on the IL-2 promoter. Thus CREM binds to the IL-2 promoter in live SLE T cells and corroborates previous conclusions made by applying shift assays (₅).

FIG. 2: Increased binding of CREM to the IL-2 promoter in SLE T cells. T cells were treated by formalin fixation, washed, lysed and sonicated. The DNA-protein complexes were immunoprecipitated with the desired antibody (anti-CREM, anti-CREB, anti-c-Jun or anti-E 47) and extracted by protein A/G agarose sepharose beads. The DNA was purified and amplified with primers flanking the IL-2 promoter including the −180 site. The primer sites (P1 and P2) and binding sites of transcription factors NF-κB, API and the CRE are indicated. DNA from 1×10⁶ cells was used per each PCR reaction. PCR products were run on a 2% agarose gel and quantified with QuantitiyOne software. The experiment is representative for 3 normal and 3 SLE patients.

The types of washing solutions, detergents for lysing the T cells and conditions for sonicating as well as additional steps for handling T cells are standard procedures known in the art and are detailed in the article entitled NF-κB Regulates the Expression of the human Complement Receptor 2 Gene, Journal of Immunology, pages 6236-6243, 0022-1767/02, (2002), incorporated herein in its entirety by reference and particularly page 6237.

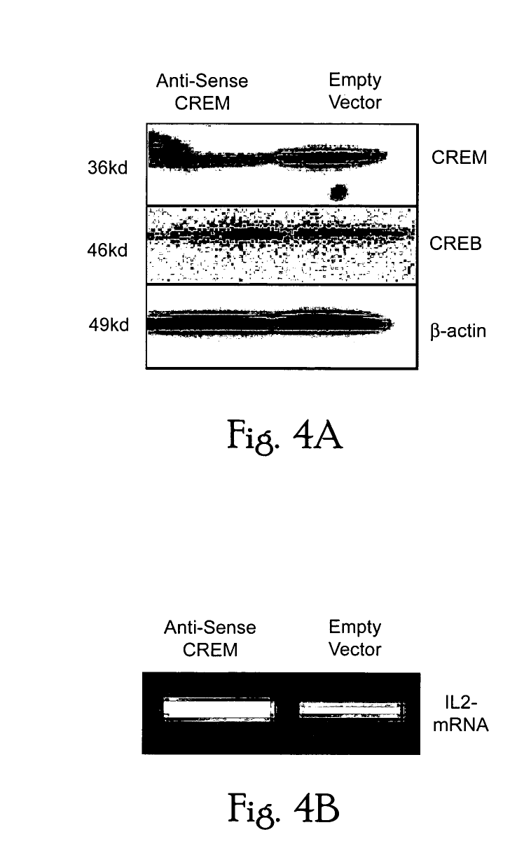

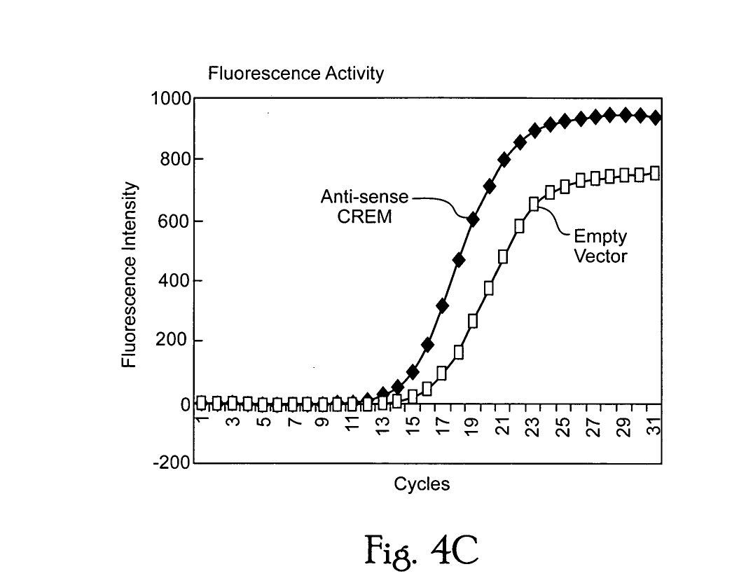

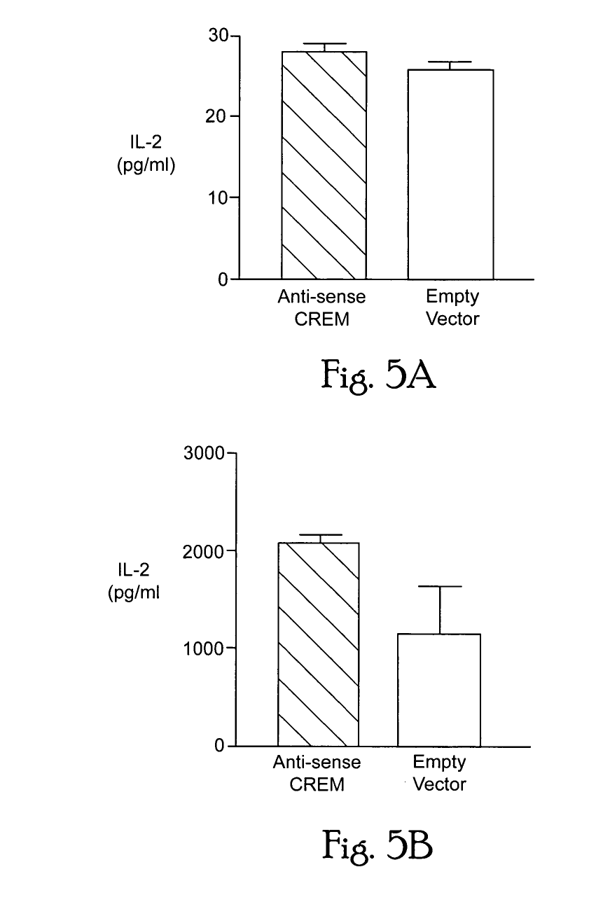

Anti-sense CREM a upregulates the activity of the IL-2 promoter. To prove that CREM α is indeed responsible for the suppression of IL-2 transcription we used sense CREM and anti-sense CREM α plasmids in SLE T cells, normal T cells and Jurkat cells. An empty vector plasmid served as control. We first used these constructs in Jurkat T cells and as it can be seen (FIG. 3) anti-sense plasmid caused increased activity of a reporter construct driven by the proximal IL-2 promoter. In contrast, the sense CREM plasmid resulted in decreased activity compared to cells transfected with the empty vector (FIG. 3). Subsequently, we asked whether transfection of SLE T cells with the anti-sense CREM plasmid would block the increased expression of CREM and restore the production of IL-2. Transfection of anti-sense CREM plasmid into SLE T cells led to decreased production of CREM protein in these cells (FIG. 4A). CREB and E actin protein levels were not affected. We noted an 8-fold upregulation of IL-2 mRNA in these cells compared to the cells transfected with an empty vector plasmid using real time PCR technique (FIG. 4B). As a result of the mRNA upregulation IL-2 protein was increased as well. The effect was especially remarkable after 6 hours stimulation with CD3 and CD28 antibody, but it was also noticeable in unstimulated T cells (FIGS. 5A and B).

FIG. 3: Anti-sense CREM plasmid increases while sense CREM decreases IL-2 promoter luciferase activity compared to an empty vector plasmid in Jurkat cells. Reporter (luciferase) construct driven by the IL-2 promoter (from −575 to +57 bp) was co-transfected with either CREM α sense, anti-sense CREM, or empty vector plasmids as described in methods. After 20 h, Jurkat cells were harvested and the luciferase assay was carried out as described in materials and methods. The picture shows mean and ±SEM of 3 experiments. Comparable results were obtained in normal T cells as well.

FIG. 4: Anti-sense CREM down regulates CREM protein and upregulates IL-2 mRNA in SLE T cells. A. SLE T cells were transfected with anti-sense CREM a (ASC) or empty vector (EV) plasmids and the CREM, CREB and E actin protein levels were evaluated in western blots. B. The levels of IL-2 mRNA were determined in the same samples using real time PCR. PCR products were visualized on 1.5% agarose gel and quantitated. The recording shows an 8-fold increase in the levels of the IL-2 mRNA in Anti-sense CREM transfected cells. The experiment was repeated in 10 SLE patients, 6 normal controls and in Jurkat cells with similar results.

FIG. 5: Antisense CREM upregulates IL-2 protein in unstimulated and stimulated T cells. A. Normal T cells were transfected with either Anti-sense CREM, Sense CREM or an empty vector and supernatant of the cell culture was harvested after 18 h for IL-2 Elisa. B. The T cells were treated like in figure A and after 18 h additionally stimulated for 6 h with CD 3 and CD 28 antibodies. Notice the difference in the IL-2 scale. FIG. A shows the mean of 6 experiments, while figure B shows the mean of 3 experiments.

Anti-sense CREM A down regulates the binding of CREM to the IL-2 promoter in vivo. To exclude non-specific effects of the anti-sense plasmid, we determined the levels of CREM binding to the IL-2 promoter after transfection of T cells with sense and anti-sense plasmids. As shown in FIG. 5A transfection of normal T cells with anti-sense CREM α resulted in decreased binding of CREM to the IL-2 promoter compared to cells transfected with empty vector plasmid; in contrast, transfection of T cells with sense CREM α plasmid increased the binding to the IL-2 promoter in live cells.

Because CHIP assays establish the binding of transcription factors to a large region of the promoter, we carried out shift assays using an oligonucleotide defined by the −180 site of the IL-2 promoter (FIG. 5B). Following transfection of T cells with the anti-sense CREM α the binding of nuclear protein to the −180 site in these assays was reduced, while transfection with sense CREM α clearly increased the binding to the −180 site compared to nuclear extracts from cells transfected with empty vector. The binding to the −180 site was specific, because it was abolished by a CREM antibody but not by an irrelevant antibody against CBF1 (FIG. 5C).

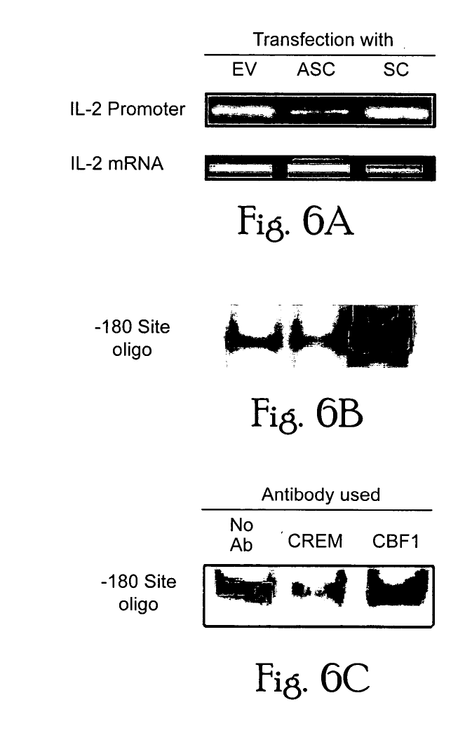

FIG. 6: Anti-sense CREM decreases and Sense CREM increases the binding of CREM to the IL-2 promoter. A. SLE T cells were transfected with either anti-sense CREM (ASC), sense-CREM (SC) or an empty vector (EV) and after 20 hours formalin fixed and sonicated. The anti-CREM Ab precipitates were subjected to PCR using IL-2 promoter specific primers. The IL-2 mRNA levels were determined using real time PCR and visualized on 1.5% agarose gel. The experiment was repeated 2 times in SLE patients and 1 time in normal T cells with similar results, although the binding of CREM in normal T cells is much lower. B. A −180 site oligonucleotide was used to determine the protein binding in normal T cells transfected as indicated above. The data shown is representative of 3 experiments in normal and 3 in SLE T cells with similar results C. The specificity of the bound protein was determined by adding the indicated antibodies to the samples from the cells transfected with the sense-CREM plasmid. The anti-CBF1 Ab was used as a non-specific control.

T cells from patients with SLE (_{8;9;21;22}) and CREB deficient mice (₇) produce decreased amounts of IL-2 in response to antigenic and mitogenic stimulation. Inability to produce IL-2 has been considered as a contributing factor for the increased infection-related morbidity and mortality in SLE patients (₉). Simultaneously, the production of IL-2 has shown to be necessary for the termination of the immune response (₁) and from that point of view, sufficient production of IL-2 is important for the control of the response to autoantigens. Defective antigen induced T cell death has been reported in human SLE T cells (_{23;24}) although the exact molecular basis of that is currently not known. Although not definitive, defective IL-2 production may explain the apparent dichotomous behaviour of SLE T cells, referred to as T cell enigma (₂₅).

Previous work from this laboratory has established that decreased IL-2 production by human SLE T cells is the consequence of defective IL-2 gene transcription due to decreased NF-κB activity (_{10;22}) as well as increased expression of the repressor CREM (⁵). Increased quantities of CREM protein were found in the nuclei of SLE T cells and the protein was shown to bind to a site 180 bp upstream of the transcription initiation point (5). This site had been previously recognised as an AP1 binding site but later it was found to bind CREM/CREB in T cells that fail to produce IL-2 (₃).

The present study has established that increased CREM expression in SLE T cell is controlled at the gene transcription level and mainly represents the CREM α isoform. The RNA stability of CREM does not differ between SLE and normal T cells. CREM was found to bind to the IL-2 promoter in live cells and therefore, unambiguously, participates in the regulation of IL-2 gene expression. The most significant finding of this study is the demonstration that an anti-sense CREM plasmid suppresses the levels of CREM mRNA and protein, its binding to the IL-2 promoter and more importantly reverses the suppressed expression of IL-2 mRNA and protein. Especially the marked increase of the IL-2 protein level after stimulation of T cells transfected with anti-sense CREM show the physiologic relevance of this finding.

The complexity of the pathogenesis of human SLE is fascinating (₂₂). On one hand someone can claim disease heterogeneity for our inability to find distinct, limited in number, pathways to account for the reported diverse cellular and cytokine abnormalities (₂₂;₂₅). On the other hand, the diverse molecular abnormalities that have been identified in SLE T cells may simply represent the expression of unique defects, the nature of which remains at large. In reference to the transcriptional repression of the IL-2 gene, a number of defects have been identified. First, the activity of NF-κB is decreased because SLE T cells lack the p65 subunit (₁₀), which after forming heterodimers with the p50 subunit accounts for increased expression of a number of genes including IL-2. SLE T cells express sufficient amounts of p50, which may homodimerize and bind to the NF-κB site of the IL-2 promoter and repress its transcriptional activity (₂₆). The origin of decreased p65 expression in SLE T cells is not known but increased caspase 8 activity, associated with the increased spontaneous apoptotic rate of SLE cells (_{27;28}), may bind and digest the p65 chain. Interestingly, forced expression of p65 reverses the decreased production of IL-2 in SLE T cells (₁₀). Second, the activity of AP1 is decreased in SLE T cells because of decreased expression of c-fos, a component of the AP1 heterodimers (₂₉). The IL-2 promoter defines a number of AP1 binding sites. Lastly, the increased expression of the repressor CREM which binds to the −180 site of the IL-2 promoter represents a central culprit in the decreased expression of IL-2. The −180 site binds CREM as shown in shift assays (_{3;5}, and FIG. 5), and in vivo (FIG. 3) and two tandem sites in front of a luciferase reporter gene are active in normal but not in SLE T cells (₅). It should be noted that in normal T cell nuclear extracts, unlike SLE T cell extracts, CREB binds to the −180 site of the IL-2 promoter. SLE T cells have decreased PKA activity (₂₁) and PKA is responsible for the activation of CREB. It is possible therefore, that defective activation of CREB may permit the expression of CREM and indirectly contribute to the suppression of the expression of IL-2. Forced expression of PKA RI beta subunit caused increased expression of IL-2 in SLE T cells (₃₀). Furthermore, it is known that c-fos contains CRE-sites in its promoter and forced expression of CREM down regulates c-fos activity (₁₃). This means that CREM can act directly on the IL-2 promoter in SLE T cells but it also influences other transcription factors that bind to the IL-2 promoter.

The demonstration (FIG. 1) of a significant, inverse correlation between IL-2 and CREM mRNA and the demonstration of direct binding of CREM to the IL-2 promoter, endows CREM with a central role in the repression of the IL-2 gene expression. The ability to re-establish the expression of IL-2 in SLE T cells by suppressing the expression of CREM with an anti-sense plasmid is of particular importance.

IL-2 infusions have been used to treat cancers including melanoma (₃₁) and renal cell carcinoma (₃₂) and they have been limited by the unwanted side effects such as capillary leak syndrome (₃₂). T cells from patients with acquired immunodeficiency syndrome fail to produce IL-2 (₃₃) and reconstitution of IL-2 production is desirable to increase the ability to generate cytotoxic responses (_{34;35}). It is possible that oligonucleotides with anti-sense CREM activity that can enter readily T cells will be designed and will be used to increase the production of IL-2 when desired. CpG oligonucleotides have been used in humans without side effects (_{36;37}) and additional modes of delivery, including liposomes, could be considered. The fact that CREM is expressed in various tissues may limit its controlled suppression but anti-sense oligonucleotides that target their effect to CREM expressed in lymphoid cells would be desirable. Herein we showed that SLE T cells mainly express the isoform CREMΔ.

In conclusion, in this study we have shown that the isoform CREM Δ bind to the IL-2 promoter and down regulate IL-2 production in SLE T-cells. Targeting the increased expression of CREM using anti-sense plasmid approaches has demonstrated a means to reverse decreased IL-2 production. Because IL-2 production is central for the ignition and termination of the immune response, the development of means to control its expression in T cells is important.

Reconstitution of Deficient TCR ζ Chain

In another embodiment of the invention, IL-2 production is improved by the reconstitution of TCR ζ chain which restores T cell signalling in SLE. The reconstitution of deficient TCR ζ chain can reverse the TCR/CD3-mediated signalling abnormalities and improve the defective IL-2 production in T cells of SLE patients. Indeed, we report that ζ chain transfection in SLE T cells can correct a number of aberrant events and restores IL-2 production.

It is well recognized that T cells from patients with systemic lupus erythematosus (SLE) display a number of T cell signaling abnormalities (38). Many of the identified molecular defects explain certain established cell and cytokine defects, whereas others should wait till additional information has been generated. We and others have established that the expression of the ζ subunit of the T cell receptor (TCR) is decreased in a majority of SLE patients (39-41) and that this defect persists over time and is independent of disease activity (52).

Despite the decreased expression of the TCR ζ chain in SLE T cells, cross-linking of the TCR/CD3 complex leads to increased free intracytoplasmic calcium concentration ([Ca²⁺]ᵢ) response (43) and protein tyrosine phosphorylation (39;41). These events appear to occur because the Fc receptor (FcR) γchain becomes a functional part of the TCR/CD3 complex (44). In support of this view is the observation that forced expression of the FcRγ renders normal T cells hyperresponsive in a manner similar to that of SLE T cells (45).

Interleukin 2 (IL-2) production in SLE T cells is decreased because of limited transcriptional activity of the IL-2 promoter due to increased expression of the repressor cAMP response element modulator (CREM) (46) and decreased expression of p65 chain of the nuclear factor (NF)-KB, which leads to decreased NF-κB activity (47). We have recently demonstrated that substitution of defective p65 subunit of NF-κB enhanced the TCR/CD3 mediated IL-2 production in SLE T cells (48).

T cells from a majority of patients with systemic lupus erythematosus (SLE) display antigen receptor-mediated signalling aberrations associated with defective T cell receptor (TCR) ζ chain, a subunit of TCR/CD3 complex. Forced expression of TCR ζ chain was found to reverse the known signalling abnormalities and defective interleukin-2 (IL-2) production in SLE T cells.

Freshly isolated SLE T cells were transfected with TCR ζ chain construct in a eukaryotic expression vector, at high efficiency by a recently developed nucleoporation technique. Restoration of TCR/CD3-mediated signalling was studied in the ζ chain transfected cells.

SLE T cells transfected with TCR ζ chain displayed increased surface expression of T cell receptor chain and normalized the TCR/CD3-induced increased free intracytoplasmic calcium concentration response as well as hyperphosphorylation of cellular substrates. Simultaneously, the previously noted increased expression of the Fc receptor &chain was diminished in SLE T cells transfected with the ζ chain expression vector and the surface membrane clusters of cell signalling molecules were redistributed to a more continuous pattern. TCR ζ chain replacement also augmented the expression of diminished TCR/CD3-mediated IL-2 production in SLE T cells associated with increased expression of the p65 subunit of the nuclear factor-KB in the nuclear fractions of SLE T cells.

These results suggest that reconstitution of deficient TCR ζ chain can reverse the TCR/CD3-mediated signalling abnormalities as well as the defective IL-2 production in T cells of SLE patients.

Materials and Methods

Patients and Controls. Patients (n=10) fulfilling the American College of Rheumatology (ACR) for the diagnosis of SLE (49) were chosen for the study. Nine patients were females and one was male; age ranged from 19 to 80 years; two were Asians, 5 were African-Americans and 3 were Caucasians; SLE disease activity index ranged from 0 (n=3) to 22 (n=2); one patient was not receiving any medications, two were receiving only hydroxychloroquine, 4 were receiving prednisone and hydroxychloroquine, whereas 3 prednisone and cytotoxic drugs. Patients who were on prednisone were asked not to take this medication at least 24 h before drawing the blood. After receiving written informed consent, heparinized peripheral venous blood sample was obtained from all participating subjects. The protocol of the study was approved by the Health Use Committees of the Uniformed Services University of the Health Sciences, the Walter Reed Army Institute of Research and Walter Reed Army Medical Center.

Antibodies. The TCR ζ chain mAb, 6B10.2, recognizing the amino acids 31-45 of the polypeptide (N-terminal mAb) was purchased from BD Pharmingen (San Diego, Calif.). The C-terminal TCR ζ chain mAb recognizing the amino acids from 145-161 and horse radish peroxidase (HRP)-conjugated anti-phosphotyrosine mAb 4G10 was purchased from Upstate Biotechnology (Lake Placid, N.Y.).

T lymphocyte isolation. Peripheral blood mononuclear cells (PBMCs) were obtained by density gradient centrifugation over Lymphoprep (Nycomed Pharma, Olso, Norway). Subsequently, T cells were isolated from PBMCs by depletion of non-T cells using a cocktail of hapten-conjugated antibodies and magnetic separation on MACS column (Miltenyi Biotec, Auburn, Calif.) as described earlier (44). In all cases, the percentage of T cells in the isolated subpopulation was >98% as determined by anti-CD3 staining and fluorescence analysis (Coulter, Hialeah, Fla.).

Cloning of TCR [symbol] chain gene in eukaryotic expression vector. The TCR ζ chain cDNA from nucleotide 34 to 563 without the stop codon was amplified by high fidelity polymerase chain reaction (PCR) system. The PCR product was cloned to pcDNA3.1/V5-HisTopo vector (Invitrogen). Plasmids were isolated from 12 colonies and restriction mapped with BamH I and Bst XI. Nucleotide sequence of clones with proper orientation was verified by DNA sequencing and used in transfection studies. TCR ζ chain constructs were also made by including the stop codon in the 3′ primer. These constructs express TCR ζ chain without V5 and His 6 peptide fusion and used to rule out the effect of these fusion components.

Transfection by nucleoporation. T cells (5-20 million) were re-suspended in 100 μl nuleofector solution and transferred to a 0.2 cm gap electroporation cuvette. The cells were mixed with 5 μg of plasmid DNA constructs and nulecoporation was performed on an Amaxa nucleoporator (Germany) using optimized program U-14 at room temperature. Transfected cells were immediately transferred to culture medium and incubated at 37° C. in 5% CO₂. (Clin Immunol. 2002 May;103(2):145-53. Direct transfer of p65 into T lymphocytes from systemic lupus erythematosus patients leads to increased levels of interleukin-2 promoter activity, Authors Herndon T M, Juang Y T, Solomou E E, Rothwell S W, Gourley M F, Tsokos G C, Department of Cellular Injury, Walter Reed Army Institute of Research, Silver Spring, Md. 20910, USA), incorporated herein in its entirety by reference. The paper describes the method and its use to effectively transfect human (normal and SLE) lymphocytes with gene expressing vectors. In this case p65.

Immunoblotting of TCR ζ chain. T cell lysates were electrophoresed and blotted and probed with the anti-TCR [symbol] chain, murine mAb 6B10.2 as previously described (50). Detergent-insoluble fractions were obtained and processed as described previously (50).

T cell activation and anti-phosphotyrosine immunoblotting. T cells were stimulated with 10 μg/ml OKT3 for 0, 1 and 2 min at 37° C. The reaction was stopped by the addition of 0.5 ml ice-cold 2× stop buffer (50 mM Tris, 100 mM NaCl, 100 mM NaF, 2 mM Na₃VO₄, 10 mM ethylenediaminetetraacetic acid (EDTA), 10 mM sodium pyrophosphate, 2 mM phenylmethanelsylfonyl fluoride (PMSF), 20 μg/ml leupeptin and 20 μg/ml aprotinin). Cells were pelleted and lysed in 1% NP-40 (Sigma chemical Co., St. Louis, Mo.) buffer as described previously (13). The lysate proteins (20 μg/lane) were analyzed in 12% SDS-PAGE gel, and immunoblotted with the anti-phosphotyrosine-hroseradish peroxidase (HRP) conjugated 4G10 mAb (1:1500) and detected using enhanced chemiluminescence (ECL) kit (Amersham Bioscineces).

Confocal microscopy. T cells were suspended in 100 μl of RPMI (Cell culture medium, Rosweli Park Memorial Institute) supplemented with 1% fetal bovine serum and adhered on poly-lysine coated slides by incubating for 1 hr at room temperature. Where necessary, cells were activated with anti-CD3-IgM (clone 2Ad2A2, a kind gift from Dr. Ellis Reinhertz, Dana Farber Cancer Institute, Boston, Mass.) at 37° C. for 2 min. The reaction was stopped with 3% paraformaldehyde for 15 min and cells were permeabilized with a buffer containing 0.05% saponin and the blocking antibody human IgG 1 μg. Cells were stained with anti-CD3 ζ (clone C-20) and anti- linker for activation of T cells (LAT, clone FL-233, Santa Cruz Biotech Inc., Santa Cruz, Calif.) for 1 hr at room temperature and counter stained with anti-goat-tetramethylrhodamine isothiocyanate (TRITC) and anti-rabbit-fluorescein isothiocyanate (FITC) (Jackson Immuno Research Labs Inc, West grove, Pa.) respectively for 30 min. Cells were washed, air dried and mounted using Gel/Mount™ (Biomeda Corp., Foster City, Calif.). Cover slips were applied and the edges sealed with clear nail polish. Samples were analyzed with laser scanning confocal fluorescence microscope (Olympus IX70 scope) with Bio-Rad Lasersharp2000 software.

Densitometry and statistical analysis. Densitometric analysis of the autoradiograms was done by software program GelPro (Media Cybernetics, Silver Spring, Md.). Statistical analysis of the data was done using the software MINITAB, Version 13 (Minitab Inc., State College, Pa.) by paired t test and P≦0.05 was considered as statistically significant.

Results

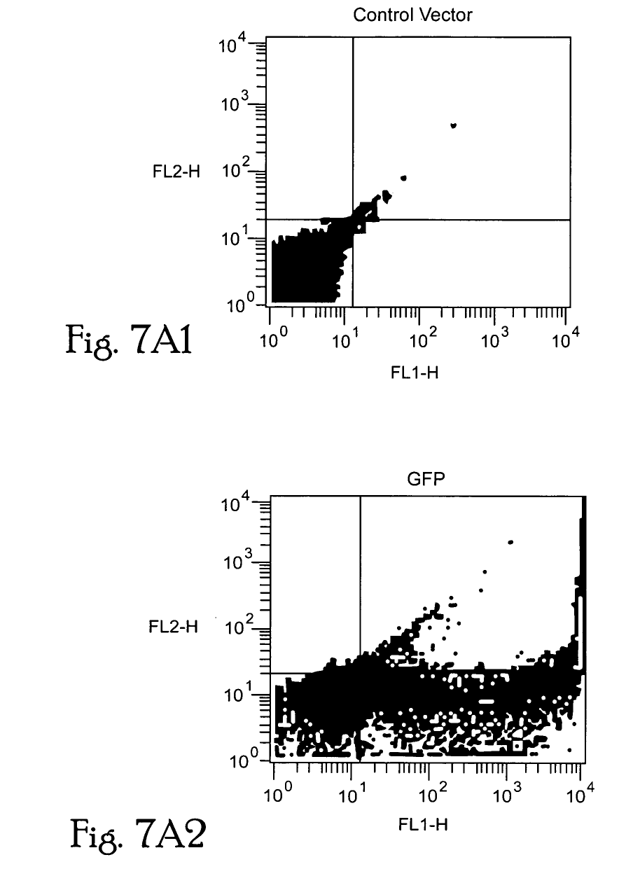

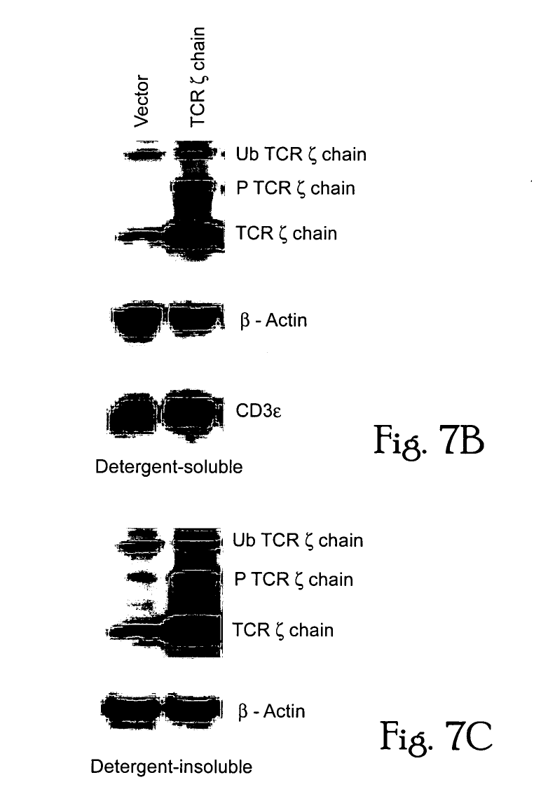

Transfection and expression of TCR] chain in SLE T lymphocytes. First we determined the efficiency of a newly developed nucleoporation protocol to transfect SLE T cells. Plasmid pIRES containing enhanced GFP was used as a positive control and a vector containing β-galactosidase gene was used as negative control. The data show that nucleoporation reproducibly results in 70-75 % transfection efficiency of GFP in SLE T cells after 18 h, which is comparable to that of normal T cells (FIG. 7A). SLE T cells were then transfected by nucleoporation with TCR ζ chain construct placed in the expression vector pCDNA 3.1/HIS-TOPO. After 18 h, the cells were lysed and the level of TCR ζ chain was measured by immunoblotting with a C-terminal specific antibody that recognizes the unmodified 16 kDa, phosphorylated p21 and 23 kDa forms and major ubiquitinated forms of the ζ chain (50). Immunoblotting results showed high expression levels of all three major forms of the ζ chain in TCR ζ chain transfected SLE T cells (FIG. 7B). Under these conditions, the expression of CD3 ε or β-actin remained similar in all samples.

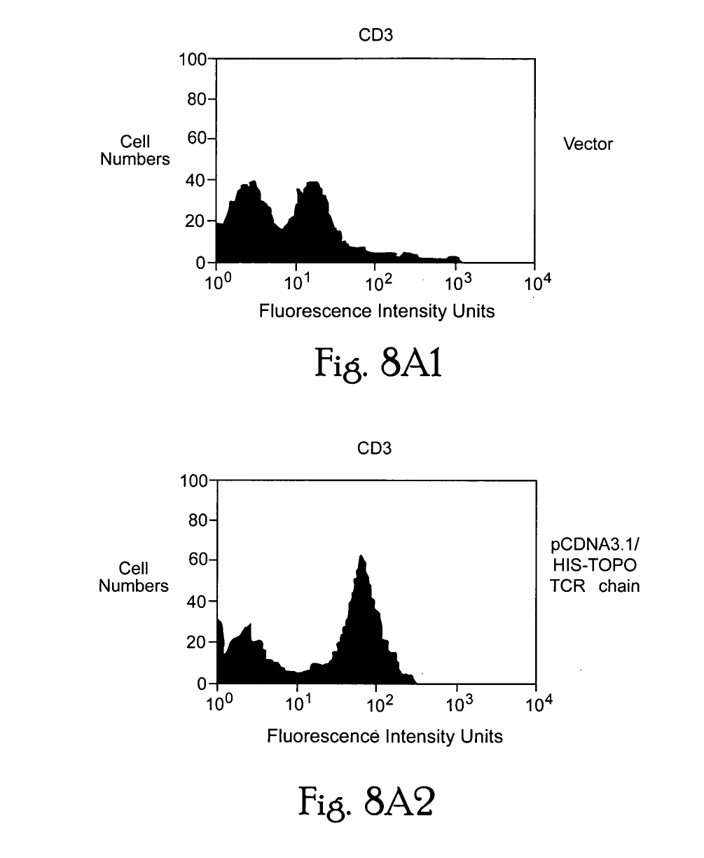

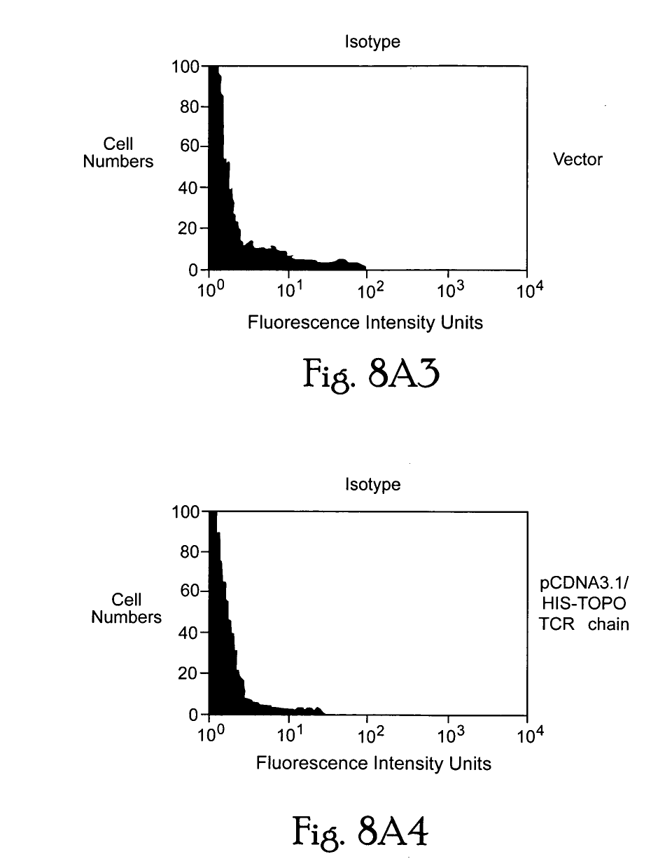

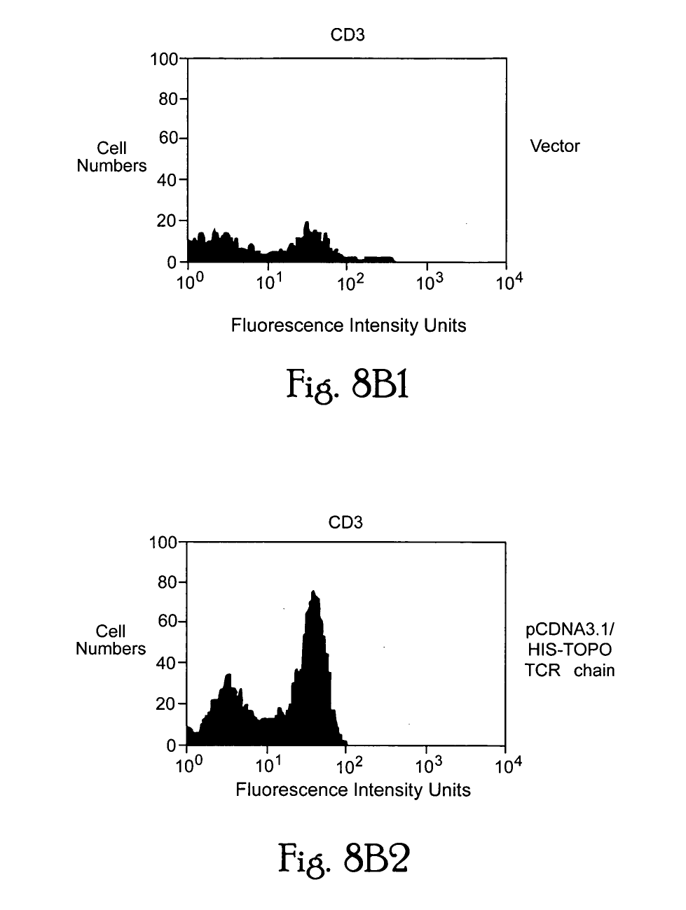

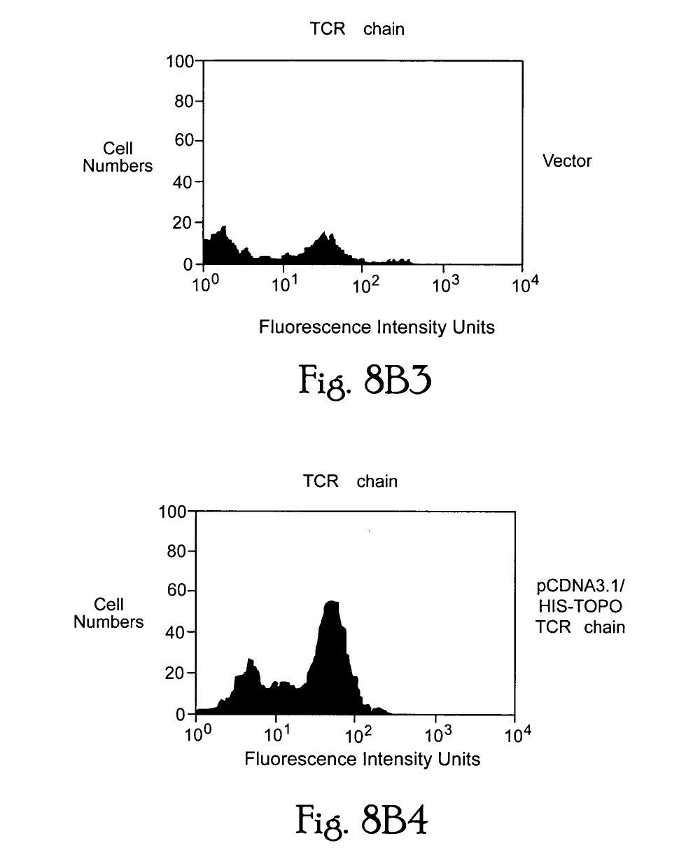

Increased surface expression of TCR/CD3 complex in TCR ζ chain transfected SLE T cells. The surface levels of TCR/CD3 are decreased in SLE T cells (51). Because TCR ζ chain is critical for the assembly, transport and surface expression of TCR/CD3 complex (52), we estimated the effect of transfection of TCR ζ chain on the levels of surface TCR/CD3 complex by fluorescence analysis. As shown in FIGS. 8a and 8b, the levels of TCR/CD3 complex are significantly increased in TCR ζ chain transfected SLE T cells. To determine whether replacement of ζ chain increased the levels of CD3 ε chain or merely facilitated its transfer to the surface membrane, we determined the levels of CD3 ε in permeabilized T cells. The data indicate that increases in ζ chain expression facilitate assembly and surface expression of the TCR/CD3 complex, rather than increasing the total levels of CD3 ε chain (FIG. 8a and 8b).

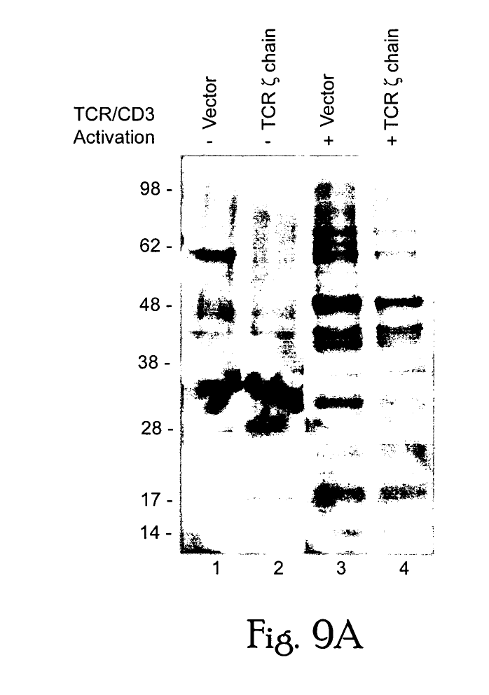

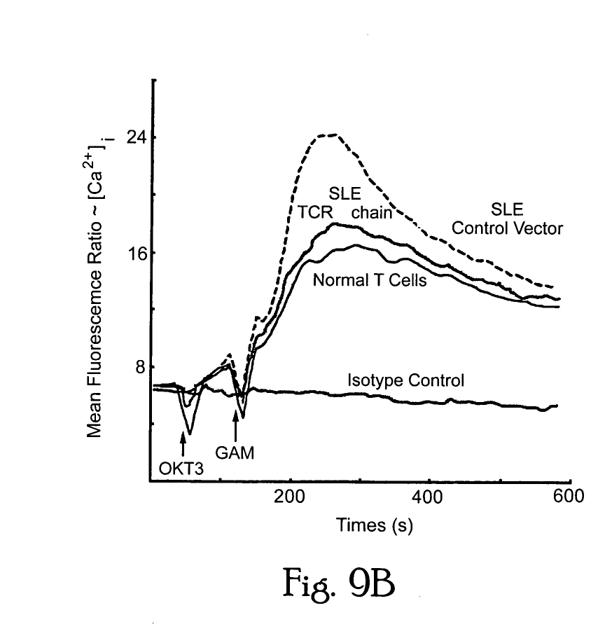

Reconstitution of TCR ζ chain expression reverses the TCR/CD3-mediated increased cytosolic protein tyrosine phosphorylation and [Ca²⁺]ᵢ response in SLE T cells. One of the hallmarks of SLE T cell signalling abnormalities is the increased TCR/CD3-mediated tyrosine phosphorylation of cellular protein substrates (2) and increased [Ca²⁺]ᵢ response. The kinetics of phosphorylation was also altered in SLE T cells with a peak phosphorylation at one min in contrast to two min in normal T cells (39). We examined the tyrosine phosphorylation of cellular protein substrates in TCR ζ chain transfected SLE T cells after activation with OKT3 antibody for 1 min. As it is shown in FIG. 9A, TCR ζ chain transfection restored the normal pattern and kinetics of tyrosine phosphorylation of cellular substrates. Similarly, transfection of SLE T cells with the ζ chain construct corrected the heightened calcium response in SLE T cells (FIG. 9B).

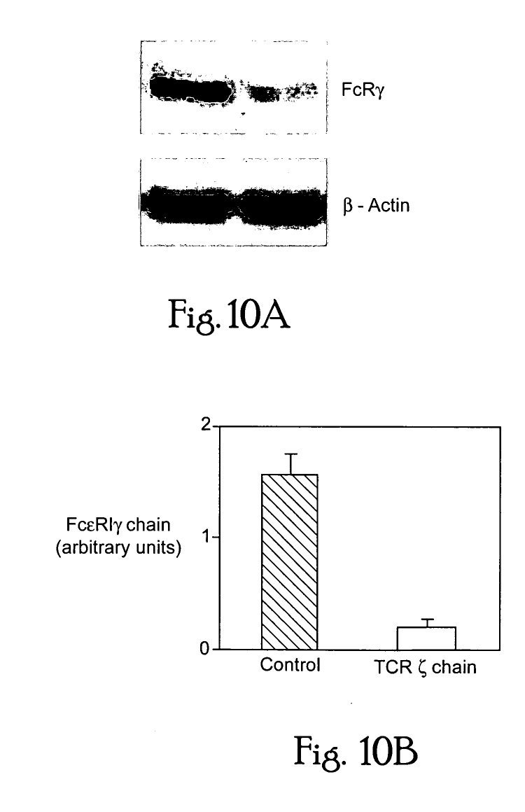

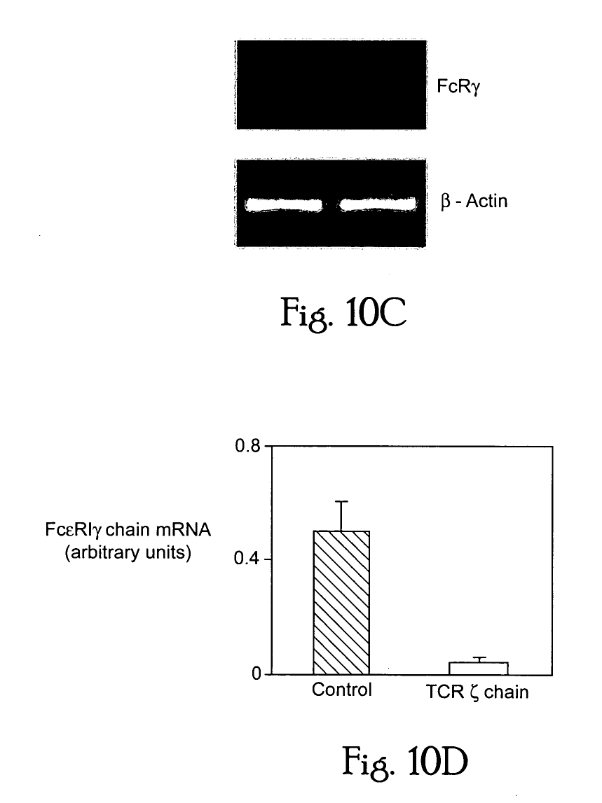

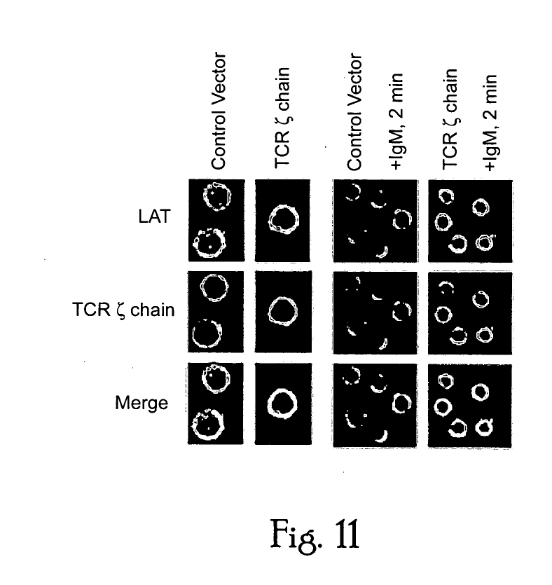

TCR ζ chain transfection down regulates FcR [symbol] expression in SLE T cells. We have shown that SLE T cells, unlike normal T cells, express FcRγ chain, which replaces the deficient TCR ζ chain and participates in T cell signaling events (44). We asked whether reconstitution of the TCR ζ chain blocked the expression of FcR [symbol] in SLE T cells. Indeed, transfection of SLE T cells with TCR ζ chain significantly reduced the expression of FcRγ chain (FIG. 10A). Decreased FcRγ protein expression is due to decreased expression of FcRγ mRNA (FIG. 10B). These data indicate that FcRγ chain is expressed only when the ζ chain is absent or decreased.

TCR ζ chain reconstitution dissolves the clustered membrane distribution of the TCR ζ chain in SLE T cells. We have recently demonstrated that the residual TCR ζ chain in SLE T cells is distributed in clusters on the surface membrane (50). These ζ chain clusters co-localize with the lipid raft markers suggesting that they represent pre-aggregated lipid rafts containing signaling molecules. Therefore, we determined the membrane distribution of the ζ chain in ζ chain-transfected and control SLE T cells by confocal microscopy. After 18 h transfection, the cells were mildly fixed, permeabilized and double-labeled with anti-ζ chain or with an Ab against the lipid raft marker LAT. The confocal microscopy data showed that, transfection with the TCR ζ chain reversed the pre-formed membrane clusters of the residual TCR ζ chain in SLE T cells and the ζ chain appeared in a uniform ring pattern (FIG. 11). Staining for the lipid raft marker LAT confirmed that pre-aggregated rafts are reversed. Interestingly, transfection with TCR ζ chain also reversed the faster kinetics of CD3 capping in SLE T cells (FIG. 11). Taken together, these data suggest that reconstitution of the TCR ζ chain normalizes the abnormal morphological distribution of signaling molecules and membrane dynamics in SLE T cells.

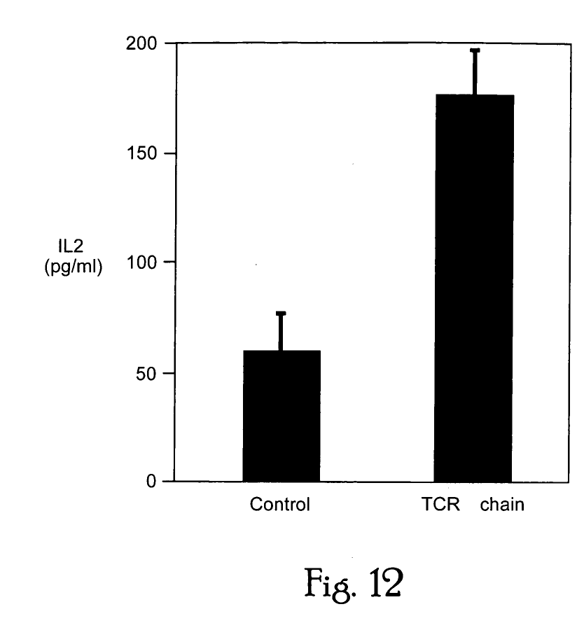

TCR ζ chain transfection augments the production of TCR/CD3-induced IL-2 in SLE T cells. SLE T cells produce less IL-2 upon activation. Decreased expression of the p65 NF-κB chain (47) has been considered responsible for the decreased transcriptional activity of the IL-2 promoter. To determine whether nucleoporation with TCR ζ chain augmented the decreased production of IL-2 in SLE T cells, we measured IL-2 levels in the supernatants of ζ chain nucleoporated SLE T cells activated with anti-CD3 and anti-CD28 mAbs. As shown in FIG. 12, the amount of IL-2 secretion significantly increased in ζ chain replenished SLE T cells. These results suggest that substitution of TCR ζ chain restores the synthesis of IL-2 in SLE T cells.

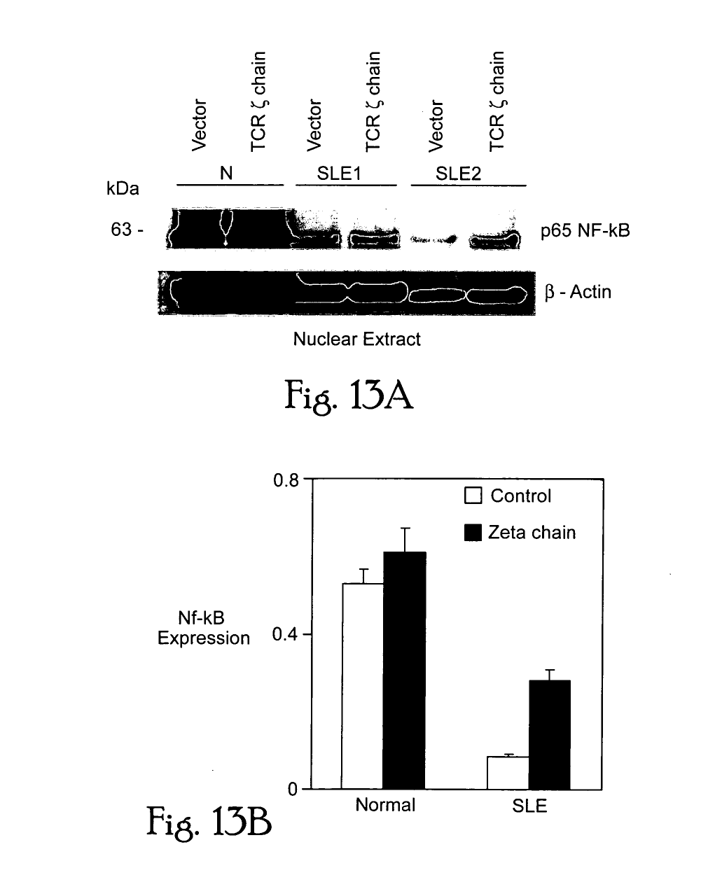

To determine whether the increased production of IL-2 was associated with restoration of the decreased expression of the p65 chain of NF-κB, we immunoblotted nuclear fraction proteins from ζ chain-transfected T cells with anti-p65 Ab. As shown in FIGS. 13a and 13b and, the level of expression of p65 NF-κB in the nuclear extracts is increased by 3-fold in SLE T cells nucleoporated with TCR ζ chain. A similar increase in the level of expression of NF-κB was also found in the nuclear fraction of ζ chain transfected SLE T cells activated with OKT3 antibody (data not shown).

Discussion

The data presented herein demonstrate that reconstitution of deficient TCR ζ chain in SLE T cells partially restores T cell signaling and function. TCR ζ chain replacement increased the surface expression of TCR/CD3 complex, normalized the TCR/CD3-mediated phosphorylation of cellular protein substrates and [Ca²⁺]ᵢ response and augmented IL-2 production. In this respect, we have demonstrated that low levels of TCR ζ chain play a substantial role in orchestrating T cell signaling abnormalities and T cell lymphokine production and that maintaining the expression levels of the TCR ζ chain is a vital component for the prevention of signaling aberrations.

Previously, Khan et al (53) have shown that bypassing a block in protein kinase A RIP enhances TCR/CD3-mediated IL-2 production. Our group has shown that reconstitution of p65 NF-κB restores CD3-mediated IL-2 production in SLE T cells (48) as does the elimination of the transcription repressor CREM by using an anti-sense CREM plasmid (54). In these approaches though, a sub optimal dose of significantly increased in ζ chain replenished SLE T cells. These results suggest that substitution of TCR ζ chain restores the synthesis of IL-2 in SLE T cells.

To determine whether the increased production of IL-2 was associated with restoration of the decreased expression of the p65 chain of NF-κB, we immunoblotted nuclear fraction proteins from ζ chain-transfected T cells with anti-p65 Ab. As shown in FIGS. 13a and 13b and, the level of expression of p65 NF-κB in the nuclear extracts is increased by 3-fold in SLE T cells nucleoporated with TCR ζ chain. A similar increase in the level of expression of NF-κB was also found in the nuclear fraction of C chain transfected SLE T cells activated with OKT3 antibody (data not shown).

Discussion

The data presented herein demonstrate that reconstitution of deficient TCR ζ chain in SLE T cells partially restores T cell signaling and function. TCR ζ chain replacement increased the surface expression of TCR/CD3 complex, normalized the TCR/CD3-mediated phosphorylation of cellular protein substrates and [Ca²⁺]ᵢ response and augmented IL-2 production. In this respect, we have demonstrated that low levels of TCR ζ chain play a substantial role in orchestrating T cell signaling abnormalities and T cell lymphokine production and that maintaining the expression levels of the TCR ζ chain is a vital component for the prevention of signaling aberrations.

Previously, Khan et al (53) have shown that bypassing a block in protein kinase A RIP enhances TCR/CD3-mediated IL-2 production. Our group has shown that reconstitution of p65 NF-κB restores CD3-mediated IL-2 production in SLE T cells (48) as does the elimination of the transcription repressor CREM by using an anti-sense CREM plasmid (54). In these approaches though, a sub optimal dose of phytohemagglutinin A was used to condition the cells for gene transfer, a process that may have altered cell physiology. The nucleoporation-based transfection technique that was used in the experiments reported in this communication does not require any cell conditioning and the transfected gene product is expressed as early as 4 hours (not shown). In addition, as shown in FIG. 7, the transfection efficiency of SLE T cells is high.

We reported previously that the residual ζ chain in SLE T cells can be found in surface membrane clusters along with LAT (50). It appears that the remaining ζ chain localizes in membrane clusters with other signaling molecules to ready the cell to respond to antigen stimulation. In addition, the expressed FcRγ chain contributes to aberrant signaling because forced expression of FcRγ chain in normal T cells leads to aberrant CD3-mediated signaling reminiscent of that observed in SLE T cells (45). Although increased degradation through the ubiquitin pathway contributes to the decreased expression of TCR ζ chain in SLE patients (45), it appears that decreased transcription of the ζ chain gene because of decreased production of active DNA-binding Elf-1 (55), is primarily responsible for the decreased production of ζ chain in SLE T cells. We cannot explain how replenishment of ζ chain upregulates the expression of the p65 chain but it is possible that it may involve phosphorylation of IκB that leads to decreased expression of its inhibitory function on NF-κB (56).

Replenishment of missing immunoregulatory molecules in autoimmune disease animal models by gene therapy approaches has been considered extensively (57). Perfection of the used vectors and of cell transfection protocols may allow the correction of signaling and cytokine defects in human disease. This study shows that effective replacement of a single key signaling molecule in SLE T cells leads not only to correction of signaling defects but also to restoration of IL-2 production. Decreased IL-2 production in SLE (58) has been associated with increased infection-related morbidity and mortality (59). It is also possible that restoration of ( chain in tumor infiltrating cells (60) will effectively increase their tumor cytotoxic ability.

Treatment of humans: We propose to leukophorese patients (remove lymhocytes with an apparatus widely used to collect pleatelets in blood banks, subject them to electroporation, whereby we will insert, a zeta chain construct and then reinfuse the cells into the patient. Similar approach will be used for the insertion of the antisense CREM.

The sequences for TCR chain, C terminal TCR ζ chain mAb, TCR/CD3 complex, P65 subunit of the nuclear factor-κB, NF-κB, IL-2, Fc receptor y chain, and CREM are all known and deposited in the NCBI (NIH) data base.

There are several types of apparatuses on the market for removing T cells from a patient and they are routinely used by blood banks. The blood will come out from one arm vein, will be centrifuged (it is a closed system) and the lymphocytes will be removed. They will be transfected with the appropriate vector and then, they will be re-administered through an arm vein. The leukapheresis apparatus delivers T cells back-into the body as well as remove them. Transfection of cells will take place under sterile conditions.

The dose range will be between 10 million cells and build up to 1 billion.

Acceptable plasmids include but are not limited to non-viral vectors—such as the ones used in the invitro experiments or sufficiently modified tretroviral vectors or sufficiently modified adnoviruses. Other harmless vectors are also acceptable.

Abbreviations:

AP 1, activating protein 1; CHIP, chromatin immuno precipitation; CRE, cAMP response element; CREB, cAMP responsive element binding protein; CREM, cAMP responsive element modulator; HRP, horse-radish peroxidase; NF-κB, nuclear factor kappa B, PKA, protein kinase A; SLE, systemic lupus erythematosus;

### Reference List

All references listed below are incorporated in their entirety herein by reference.

1. Refaeli, Y., L. Van Parijs, C. A. London, J. Tschopp, and A. K. Abbas. 1998. Biochemical mechanisms of IL-2-regulated Fas-mediated T cell apoptosis. Immunity. 8:615-623.

2. Jain, J., C. Loh, and A. Rao. 1995. Transcriptional regulation of the IL-2 gene. Curr. Opin. Immunol. 7:333-342.

3. Powell, J. D., C. G. Lerner, G. R. Ewoldt, and R. H. Schwartz. 1999. The −180 site of the IL-2 promoter is the target of CREB/CREM binding in T cell anergy. J. Immunol. 163:6631-6639.

4. Rothenberg, E. V. and S. B. Ward. 1996. A dynamic assembly of diverse transcription factors integrates activation and cell-type information for interleukin 2 gene regulation. Proc. Natl. Acad. Sci. U.S.A 93:9358-9365.

5. Solomou, E. E., Y. T. Juang, M. F. Gourley, G. M. Kammer, and G. C. Tsokos. 2001. Molecular basis of deficient IL-2 production in T cells from patients with systemic lupus erythematosus. J. Immunol. 166:4216-4222.

6. Solomou, E. E., Y. T. Juang, and G. C. Tsokos. 2001. Protein kinase C-theta participates in the activation of cyclic AMP-responsive element-binding protein and its subsequent binding to the −180 site of the IL-2 promoter in normal human T lymphocytes. J. Immunol. 166:5665-5674.

7. Barton, K., N. Muthusamy, M. Chanyangam, C. Fischer, C. Clendenin, and J. M. Leiden. 1996. Defective thymocyte proliferation and IL-2 production in transgenic mice expressing a dominant-negative form of CREB. Nature 379:81-85.

8. Garcia-Cozar, F. J., I. J. Molina, M. J. Cuadrado, M. Marubayashi, J. Pena, and M. Santamaria. 1996. Defective B7 expression on antigen-presenting cells underlying T cell activation abnormalities in systemic lupus erythematosus (SLE) patients. Clin. Exp. Immunol. 104:72-79.

9. Via, C. S., G. C. Tsokos, B. Bermas, M. Clerici, and G. M. Shearer. -1993. T cell-antigen-presenting cell interactions in human systemic lupus erythematosus. Evidence for heterogeneous expression of multiple defects. J. Immunol. 151:3914-3922.

10. Wong, H. K., G. M. Kammer, G. Dennis, and G. C. Tsokos. 1999. Abnormal NF-kappa B activity in T lymphocytes from patients with systemic lupus erythematosus is associated with decreased p65-RelA protein expression. J. Immunol. 163:1682-1689.

11. Hussain, M. A., P. B. Daniel, and J. F. Habener. 2000. Glucagon stimulates expression of the inducible cAMP early repressor and suppresses insulin gene expression in pancreatic beta-cells. Diabetes 49:1681-1690.

12. Foulkes, N. S., G. Duval, and P. Sassone-Corsi. 1996. Adaptive inducibility of CREM as transcriptional memory of circadian rhythms. Nature 381:83-85.

13. Foulkes, N. S., B. M. Laoide, F. Schlotter, and P. Sassone-Corsi. 1991. Transcriptional antagonist cAMP-responsive element modulator (CREM) down-regulates c-fos cAMP-induced expression. Proc. Natl. Acad. Sci. U.S.A 88:5448-5452.

14. Daniel, P. B., L. Rohrbach, and J. F. Habener. 2000. Novel cyclic adenosine 3′,5′-monophosphate (cAMP) response element modulator theta isoforms expressed by two newly identified cAMP-responsive promoters active in the testis. Endocrinology 141:3923-3930.

15. De Cesare, D. and P. Sassone-Corsi. 2000. Transcriptional regulation by cyclic AMP-responsive factors. Prog. Nucleic Acid Res. Mol. Biol. 64:343-369.

16. Monaco, L. and P. Sassone-Corsi. 1997. Cross-talk in signal transduction: Ras-dependent induction of cAMP-responsive transcriptional repressor ICER by nerve growth factor. Oncogene 15:2493-2500.

17. Sassone-Corsi, P. 1995. Transcription factors responsive to cAMP. Annu. Rev. Cell Dev. Biol. 11:355-377.

18. Bodor, J., L. Feigenbaum, J. Bodorova, C. Bare, M. S. Reitz, Jr., and R. E. Gress. 2001. Suppression of T-cell responsiveness by inducible cAMP early repressor (ICER). J. Leukoc. Biol. 69:1053-1059.

19. Bodor, J., J. Bodorova, and R. E. Gress. 2000. Suppression of T cell function: a potential role for transcriptional repressor ICER. J. Leukoc. Biol. 67:774-779.

20. Bombardier, C., D. D. Gladman, M. B. Urowitz, D. Caron, and C. H. Chang. 1992. Derivation of the SLEDAI. A disease activity index for lupus patients. The Committee on Prognosis Studies in SLE. Arthritis Rheum. 35:630-640.

21. Kammer, G. M., T. M. Haqqi, P. Hasler, and C. J. Malemud. 1993. The effect of circulating serum factors from patients with systemic lupus erythematosus on protein kinase A (PKA) activity and PKA-dependent protein phosphorylation in T lymphocytes. Clin. Immunol. Immunopathol. 67:8-16.

22. Tsokos, G. C., H. K. Wong, E. J. Enyedy, and M. P. Nambiar. 2000. Immune cell signaling in lupus. Curr. Opin. Rheumatol. 12:355-363.

23. Kovacs, B., S. N. Liossis, G. J. Dennis, and G. C. Tsokos. 1997. Increased expression of functional Fas-ligand in activated T cells from patients with systemic lupus erythematosus. Autoimmunity 25:213-221.

24. Kovacs, B., D. Vassilopoulos, S. A. Vogelgesang, and G. C. Tsokos. 1996. Defective CD3-mediated cell death in activated T cells from patients with systemic lupus erythematosus: role of decreased intracellular TNF-alpha. Clin. Immunol. Immunopathol. 81:293-302.

25. Dayal, A. K. and G. M. Kammer. 1996. The T cell enigma in lupus. Arthritis Rheum. 39:23-33.

26. Burgos, P., C. Metz, P. Bull, R. Pincheira, L. Massardo, C. Errazuriz, M. R. Bono, S. Jacobelli, and A. Gonzalez. 2000. Increased expression of c-rel, from the NF-kappaB/Rel family, in T cells from patients with systemic lupus erythematosus. J. Rheumatol. 27:116-127.

27. Takeda, Y., P. Caudell, G. Grady, G. Wang, A. Suwa, G. C. Sharp, W. S. Dynan, and J. A. Hardin. 1999. Human RNA helicase A is a lupus autoantigen that is cleaved during apoptosis. J. Immunol. 163:6269-6274.

28. Horiuchi, T., D. Himeji, H. Tsukamoto, S. Harashima, C. Hashimura, and K. Hayashi. 2000. Dominant expression of a novel splice variant of caspase-8 in human peripheral blood lymphocytes. Biochem. Biophys. Res. Commun. 272:877-881.

29. Alvarado-de la Barrera, C., J. Alcocer-Varela, Y. Richaud-Patin, D. Alarcon-Segovia, and L. Llorente. 1998. Differential oncogene and TNF-alpha mRNA expression in bone marrow cells from systemic lupus erythematosus patients. Scand. J. Immunol. 48:551-556.

30. Khan, I. U., D. Laxminarayana, and G. M. Kammer. 2001. Protein kinase A RI beta subunit deficiency in lupus T lymphocytes: bypassing a block in RI beta translation reconstitutes protein kinase A activity and augments IL-2 production. J.Immunol. 166:7600-7605.

31. Nathan, P. D. and T. G. Eisen. 2002. The biological treatment of renal-cell carcinoma and melanoma. Lancet Oncol. 3:89-96.

32. Sule, N. S., R. P. Nerurkar, and S. Kamath. 2001. Interleukin-2 as a therapeutic agent. J. Assoc. Physicians India 49:897-900.

33. Meyaard, L., H. Schuitemaker, and F. Miedema. 1993. T-cell dysfunction in HIV infection: anergy due to defective antigen-presenting cell function? Immunol. Today 14:161-164.

34. Mitsuyasu, R. T. 2001. The potential role of interleukin-2 in HIV. AIDS 15 Suppl 2:S22-S27.

35. Kinter, A. and A. S. Fauci. 1996. Interleukin-2 and human immunodeficiency virus infection: pathogenic mechanisms and potential for immunologic enhancement. Immunol. Res. 15:1-15.

36. Krieg, A. M. 2000. Immune effects and mechanisms of action of CpG motifs. Vaccine 19:618-622.

37. Sin, J. I. and D. B. Weiner. 2000. Improving DNA vaccines targeting viral infection. Intervirology 43:233-246.

38. Kammer G M, Perl A, Richardson B C, Tsokos G C. Abnormal T cell signal transduction in systemic lupus erythematosus. Arthritis Rheum. 2002; 46:1139-54.

39. Liossis S N, Ding D Z, Dennis G J, Tsokos G C. Altered pattern of TCR/CD3-mediated protein-tyrosyl phosphorylation in T cells from patients with systemic lupus erythematosus. Deficient expression of the T-cell receptor zeta chain. J Clin Invest 1998; 101:1448-57.

40. Tsuzaka K, Takeuchi T, Onoda N, Pang M, Abe T. Mutations in T cell receptor zeta chain mRNA of peripheral T cells from systemic lupus erythematosus patients. J Autoimmun. 1998;11:381-5.

41. Brundula V, Rivas L J, Blasini A M, et al. Diminished levels of T cell receptor zeta chains in peripheral blood T lymphocytes from patients with systemic lupus erythematosus. Arthritis Rheum. 1999; 42:1908-16.

42. Nambiar, M. P., Mitchell, J. P., Ceruti, M. A., Malloy, M. A, and Tsokos, G. C. Prevalence of T cell receptor zeta chain deficiency in systemic lupus erythematosus. Lupus in press, 2002.

43. Vassilopoulos D, Kovacs B, Tsokos G C. TCR/CD3 complex-mediated signal transduction pathway in T cells and cell lines from patients with systemic lupus erythematosus. J Immunol 1995; 155:2269-81.

44. Enyedy E J, Nambiar M P, Liossis S N, Dennis G, Kammer G M, Tsokos G C. Fc epsilon receptor type I gamma chain replaces the deficient T cell receptor zeta chain in T cells of patients with systemic lupus erythematosus. Arthritis Rheum 2001; 44:1114-21.

45. Nambiar, M. P., Fisher, C. U., Kumar, A., Tsokos, C. G., Warke, V. G., and Tsokos, G. C. Forced expression of the Fc receptor chain renders human T cells hyperresponsive to antigen stimulation. J. Immunol. In press. J Immunol. Accepted with revisions. 2002.

46. Solomou E E, Juang Y T, Gourley M F, Kammer G M, Tsokos G C. Molecular Basis of Deficient IL-2 Production in T Cells from Patients with Systemic Lupus Erythematosus. J Immunol 2001; 166:4216-22.

47. Wong H K, Kammer G M, Dennis G, Tsokos G C. Abnormal NF-B activity in T lymphocytes from patients with systemic lupus erythematosus is associated with decreased p65-relA protein expression. J Immunol 1999; 163:1682-9.

48. Herndon T M, Juang Y T, Solomou E E, Rothwell S W, Gourley M F, Tsokos G C. Direct transfer of p65 into T lymphocytes from systemic lupus erythematosus patients leads to increased levels of interleukin-2 promoter activity. Clin Immunol 2002; 103:145-53.

49. Tan E M, Cohen A S, Fries J F, et al. The 1982 revised criteria for the classification of systemic lupus erythematosus. Arthritis Rheum 1982; 25:1271-1277.

50. Nambiar M P, Enyedy E J, Fisher C U, et al. Abnormal expression of various molecular forms and distribution of T cell receptor zeta chain in patients with systemic lupus erythematosus. Arthritis Rheum. 2002; 46:163-74.

51. Raziuddin S, al Janadi M A, Alwabel A A. T-cell receptor alpha/beta chain-CD3 protein complex defect in systemic lupus erythematosus: T-cell function. Am J Med 1992; 93:461-466.

52. Sussman J J, Bonifacino J S, Lippincott-Schwartz-J, et al. Failure to synthesize the T cell CD3-zeta chain: structure and function of a partial T cell receptor complex. Cell 1988; 52:85-95.

53. Khan I U, Laxminarayana D, Kammer G M. Protein kinase A RI beta subunit deficiency in lupus T lymphocytes: bypassing a block in RI beta translation reconstitutes protein kinase A activity and augments IL-2 production. J Immunol 2001; 166:7600-7605.

54. Tenbrock, K., Juang, Y-T., Gourley, M. F., Nambiar, M. P., and Tsokos, G. C. Antisense cyclic adenosine 5′-monophosphate response element modulator up-regulates IL-2 in T cells from patients with systemic lupus erythematosus. J Immunol 2002; 169:4147-4152.

55. Juang, Y-T., Tenbrock, K., Nambiar, M. P., Gourley, M. F., and Tsokos, G. C. Defective production of the 98 kDa form of Elf-1 is responsible for the decreased expression of TCR zeta chain in patients with systemic lupus erythematosus. J Immunol 2002. 169: 6048-6055.

56. Li Q, Verma I M. NF-kappaB regulation in the immune system. Nat Rev Immunol 2002;2:725-734.

57. Tsokos G C, Nepom G T. Gene therapy in the treatment of autoimmune diseases. J Clin Invest 2000;106:181-183.

58. Linker-Israeli M, Bakke A C, Kitridou R C, Gendler S, Gillis S, Horwitz D A. Defective production of interleukin 1 and interleukin 2 in patients with systemic lupus erythematosus (SLE). J Immunol 1983; 130:2651-2555.

59. Iliopoulos A G, Tsokos G C. Immunopathogenesis and spectrum of infections in systemic lupus erythematosus. Semin Arthritis Rheum 1996; 25:318-36.

60. Mizoguchi H, O'Shea J J, Longo D L, Loeffler C M, McVicar D W, Ochoa A C. Alterations in signal transduction molecules in T lymphocytes from tumor-bearing mice. Science 1992;258:1795-8.

What is claimed is:

1. A method of a treating systemic lupus erythematosus patient comprising:
administering gene-modified T cells having suppressed expression of cAMP response element modulator to said systemic lupus erythematosus patient, thereby increasing the expression of IL-2.

2. The method of claim 1, wherein said gene modified T cells are from-said patient.

3. The method of claim 1, wherein said T cells having suppressed expression of cAMP response element modulator, have been treated with antisense CREM.

4. The method of claim 1, further comprising transfecting said T cells with TCR ζ chain to further increase the expression of IL-2.

5. The method of claim 1, wherein said patient is a human.

6. A method of treating systemic lupus erythematosus T cells comprising:
(a) administering anti-sense cAMP response element modulator to T cells from a patient with systemic lupus erythematosus to prevent cAMP response element modulator from binding to an IL-2 promotor in said T cell; and
(b) increasing the production of IL-2 in said T cells.

7. The method of claim 6, further comprising: after step (b) reinserting said T cells into said patient.

8. The method of claim 6, wherein said anti-sense cAMP response element modulator is administered in the form of an anti-sense cAMP response element modulator plasmid.

9. The method of claim 6, wherein said antisense cAMP response element modulator, is α-antisense cAMP response element modulator.

10. A method of treating a patient with systemic lupus erythematosus comprising:
a) removing lymphocytes from a patient having systemic lupus erythematosus;
b) leukopheresing said lymphocytes;
c) transfecting said lymphocytes with plasmid vectors containing anti-sense cAMP response element modulator; and
d) re-infusing said transfected lymphocytes into the patient.

11. The method of claim 10, wherein said antisense cAMP response element modulator, is α-antisense cAMP response element modulator.

12. A method of treating systemic lupus erythematosus T cells to increase the level of IL-2 mRNA therein comprising: decreasing the amount of cAMP response element modulator mRNA.

13. The method of claim 12, wherein the cAMP response element modulator mRNA is decreased by inserting antisense cAMP response element modulator into said T cell.

14. The method of claim 12, wherein in said cAMP response element modulator mRNA, is α cAMP response element modulator mRNA.

15. The method of treating a human patient having systemic lupus erythemotosus comprising administering said T cells that have decreased cAMP response element modulator mRNA to said patient.

16. A method of increasing IL-2 production in T cells from a patient with systemic lupus erythematosus comprising:
administering anti-sense cAMP response element modulator to T cells extracted from a patient with systemic lupus erythematosus; and preventing cAMP response element modulator from binding to an IL-2 promotor in said T cell, thereby increasing the production of IL-2.

17. The method of claim 16, wherein said patient is a human.

18. A method of treating systemic lupus erythematosus comprising:
a) removing T cells from a patient having systemic lupus erythematosus;
b) transfecting said T cells with TCR ζ chain;
c) reinserting said T cells into said patient.

19. The method of claim 18, wherein said T cells are transfected by nucleoporation.

20. The method of claim 18, wherein after said T cells are removed from said patient, mixing said cells with a plasmid DNA construct.

21. The method of claim 18, further comprising incubating said T cells after they are transfected.

22. A method of treating T cells from patients having systemic lupus erythematosus to restore T cell signaling and augment TCR/CD3-induced interleukin-2 production comprising:
a) removing and isolating T cells from a patient having systemic lupus erythematosus;
b) transfecting said T cells with a TCR ζ chain contained in a vector, wherein said transfecting is by nucleoporation;
c) incubating said T cells; and
d) restoring said T cells signaling and augmenting TCR/CD3-induced interleukin-2 production.

23. The method of claim 22, wherein said incubating is at 37° C. in 5% CO₂.

24. The method of claim 22, wherein said vector is a pcDNA3.1/V5-HisTopo vector.

25. The method of claim 22, wherein said patient is a human.

26. A method of a treating systemic lupus erythematosus patient comprising:
a) removing T cells from said patient;
b) treating said T cells from said patient with antisense cAMP response element modulator to suppress the expression of cAMP response element modulator to increase the expression of IL-2;
c) transfecting said T cells with TCR ( chain to further increase the expression of IL-2; and
d) administering said treated and gene-modified T cells to said systemic lupus erythematosus patient, thereby increasing the expression of IL-2.

27. The method of treating systemic lupus erythematosis patient's T cells comprising: reducing cAMP response element modulator α from binding to an −180 site of an IL-2 promoter in said T cells by transfecting said T cells with the anti-sense cAMP response element modulator α.

28. The method of claim 27, further comprising administering said transfected T cells to said patient intravenously.


SEQUENCE LISTING (8 sequences)

SEQ ID NO: 1 - DNA, 20 bp, Artificial Sequence
  Description of Artificial Sequence Synthetic primer
catgggtcagaaggattcct

SEQ ID NO: 2 - DNA, 19 bp, Artificial Sequence
  Description of Artificial Sequence Synthetic primer
agctggtagctcttctcca

SEQ ID NO: 3 - DNA, 27 bp, Artificial Sequence
  Description of Artificial Sequence Synthetic primer
cactactcacattaacctcaactcctg

SEQ ID NO: 4 - DNA, 29 bp, Artificial Sequence
  Description of Artificial Sequence Synthetic primer
ctgggaagcacttaattatcaagttagtg

SEQ ID NO: 5 - DNA, 30 bp, Artificial Sequence
  Description of Artificial Sequence Synthetic primer
gaaacagttgaatcccagcatgatggaagt

SEQ ID NO: 6 - DNA, 24 bp, Artificial Sequence
  Description of Artificial Sequence Synthetic primer
tgccccgtgctagtctgatatatg

SEQ ID NO: 7 - DNA, 21 bp, Artificial Sequence
  Description of Artificial Sequence Synthetic primer
ctaagtgtgggctaatgtaac

SEQ ID NO: 8 - DNA, 17 bp, Artificial Sequence
  Description of Artificial Sequence Synthetic primer
tgtaaaactgtgggggt